(19) United States
(12) Patent Application Publication
(10) Pub. No.: US 2025/0255937 A1
(43) Pub. Date: Aug. 14, 2025

(54) PRE-FILLED MANUAL INJECTOR APPARATUS

(71) Applicant: Mallinckrodt Pharmaceuticals Ireland Limited, Dublin (IE)

(72) Inventors: Susan O'Neill, Hazelwood, MO (US); Conor Briody, Dublin (IE); Simon Ryan, Hazelwood, MO (US); Sheila Elliott, St. Louis, MO (US); Johanna Purcell, Houston, TX (US)

(21) Appl. No.: 19/169,718

(22) Filed: Apr. 3, 2025

Related U.S. Application Data
(63) Continuation-in-part of application No. 18/480,264, filed on Oct. 3, 2023.
Application No. 18/480,264 is a continuation of application No. PCT/US22/75694, filed on Aug. 30, 2022.
(60) Provisional application No. 63/238,543, filed on Aug. 30, 2021.

(51) Int. Cl. A61K 38/35; A61M 5/20

(52) U.S. Cl. CPC A61K 38/35; A61M 5/2033

(57) ABSTRACT

Provided herein are methods of administering a repository corticotropin pharmaceutical composition in a pre-filled manual apparatus for an adult patient ≥18 years old or a pediatric patient ≥2 years of age by an adult of at least 18 years of age to deliver a one-time use dose only. The manual injector apparatus is capable of being stored at room temperature for up to 24 hours prior to administration and the repository corticotropin pharmaceutical composition is a naturally sourced complex mixture comprising N-25 deamidated porcine ACTH (1-39).

### CROSS-REFERENCE TO RELATED APPLICATIONS

This application is a continuation-in-part of U.S. application Ser. No. 18/480,264, filed on Oct. 3, 2023, which is a continuation of PCT Application No. PCT/US22/75694, filed on Aug. 30, 2022, which claims priority to U.S. Provisional Application No. 63/238,543, filed on Aug. 30, 2021, which are incorporated herein by reference in their entirety.

### REFERENCE TO SEQUENCE LISTING

This application contains a Sequence Listing that has been submitted in Extensible Markup Language (.xml) and is hereby incorporated by reference in its entirety. The XML copy, created Aug. 30, 2022, is named H-AD-00018-WO-724731.xml, and is 4,096 bytes.

### BACKGROUND OF THE INVENTION

Pharmaceutical compositions with thicker viscosities (e.g., corticotropins) are more difficult to inject via syringe, especially for a patient to self-inject. Due to the greater forces required to self-inject the thicker viscosity compositions, there has been an unaddressed problem of breakage of high viscosity injection devices and/or patients receiving only a partial dose. Therefore, there is a great need for new, more effective apparatuses and method for consistent manual injection of full dosages of corticotropin compositions with higher viscosities.

### SUMMARY OF THE INVENTION

Provided herein is a method comprising removing a pre-filled manual apparatus from a refrigerator, the manual apparatus comprises a repository corticotropin pharmaceutical composition for a patient ≥2 years old in need thereof; and administering the repository corticotropin pharmaceutical composition to the patient between 30 minutes and up to 24 hours after removal from the refrigerator; wherein the repository corticotropin pharmaceutical composition is administered by applying a force to the manual apparatus; wherein the repository corticotropin pharmaceutical composition is a naturally sourced complex mixture comprising N-25 deamidated porcine ACTH (1-39); wherein the repository corticotropin pharmaceutical composition has a viscosity of 5 cPs to 30 cPs and a temperature of 22° C. to 25° C. at the time of the administering; wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.; wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.; wherein the method results in a breakage rate of less than 30% during administration; and wherein the composition is consistently administered between 4 to 8 mm below the surface of the skin of the patient.

Also provided herein is a method of consistent administration of a repository corticotropin pharmaceutical composition comprising removing a pre-filled manual apparatus from a refrigerator, the manual apparatus comprising a repository corticotropin pharmaceutical composition for a patient ≥2 years old in need thereof; and waiting between 45 minutes and up to 24 hours after apparatus removal from the refrigerator; wherein the repository corticotropin pharmaceutical composition is administered by applying a force to the manual apparatus; wherein the repository corticotropin pharmaceutical composition is administered consistently at a skin depth of 4 mm to 8 mm; wherein the repository corticotropin pharmaceutical composition has a viscosity of 5 cPs to 30 cPs and a temperature of 22° C. to 25° C. at the time of the administering; wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.; wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.; and wherein the composition is consistently administered at a skin depth of 4 to 8 mm with every performed administration.

### BRIEF DESCRIPTION OF THE DRAWINGS

Implementations of the present technology will now be described, by way of example only, with reference to the attached figures, wherein:

FIG. 1 is a graph of temperature vs. max force for a 30 minute warming time.

FIG. 2 is a graph of temperature vs. force (with incompletes) for cold devices.

FIG. 3 is a sample injection force graph for a patient that shows a skewed force pattern.

FIG. 4 is a sample injection force graph for a patient that shows an oscillatory skewed force pattern.

FIG. 5 is a sample injection force graph for a patient that shows oscillations to no force exerted.

FIG. 6 is a graph of injection time vs. max force for a 23 gauge needle.

FIG. 7 is a graph of injection time vs. max force for a 25 gauge needle.

FIG. 8 is a graph of the measured viscosity of the repository corticotropin injection.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure is related to methods of administering using a pre-filled manual injector apparatus or device. Pre-filled injector devices may be designed to be easy to use and are intended to provide quick and accurate administration of medication. Various pre-filled injection devices may be configured as pre-filled manual injectors or pneumatic injectors. Pre-filled injector devices have been shown to be safe and effective for various conditions (e.g., of anaphylaxis). Pre-filled injector devices may specifically be designed to reduce the risk of user error, which can be particularly important in patient populations where patients may have trouble self-injecting with a traditional syringe system. Pre-filled injector devices may allow for consistent appropriate dosing and administration technique. In addition, pre-filled injector devices may have built-in safety features, e.g., needle shields and locking mechanisms, to prevent accidental needlestick injuries and to ensure proper dosing and injection technique.

Pre-filled injector devices may be designed to be easy to use and are intended to administer a consistent accurate dosage of a medication, and may eliminate the need for patient management of ampoules, syringes and needles.

The use of pre-filled manual injection devices in adults and pediatric patients can provide a consistent and reliable method of administering medications, especially for adults and children with chronic conditions that require regular injections. The use of a pre-filled manual injector in children may provide a consistent and reliable method of administering medication, which may be particularly important for children with chronic conditions that require regular injections.

Provided herein, in some embodiments, is a pre-filled manual injector apparatus comprising a repository corticotropin injection for an adult patient ≥18 years old in need thereof to deliver a one-time use dose only. The manual injector apparatus is capable of being stored at room temperature for up to 24 hours prior to administration. The pre-filled manual injector apparatus may be stored at a temperature between 2° C. and 8° C. and then warmed at room temperature for 30 minutes to 24 hours prior to administration.

Further provided herein, in an embodiment, is a method comprising: removing a pre-filled manual injector apparatus from a refrigerator, the manual injector apparatus comprising a repository corticotropin injection for an adult patient ≥18 years old in need thereof; and administering the repository corticotropin injection to the patient between 30 minutes and up to 24 hours after removal from the refrigerator.

Advantageously, in some embodiments, the pre-filled manual injector apparatus is capable of withstanding a force needed to self-inject a full dose of the repository corticotropin injection at a temperature between 2° C. and 25° C.

Provided herein, also, in some embodiments, is a method comprising: removing a pre-filled manual apparatus from a refrigerator, the manual apparatus comprising a repository corticotropin pharmaceutical composition for a patient ≥2 years old in need thereof; waiting between 45 minutes and up to 24 hours after apparatus removal from the refrigerator; and administering the repository corticotropin pharmaceutical composition to the patient at a consistent skin depth of 4 mm to 8 mm. In some embodiments, the repository corticotropin pharmaceutical composition may be administered by applying a force to the manual apparatus. In some embodiments, the repository corticotropin pharmaceutical composition may be a naturally sourced complex mixture comprising N-25 deamidated porcine ACTH (1-39). In some embodiments, the repository corticotropin pharmaceutical composition may have a viscosity of 5 cPs to 30 cPs and a temperature of 22° C. to 25° C. at the time of the administering. In some embodiments, the force required to administer a dose of 40 USP units/0.5 mL of the repository corticotropin pharmaceutical composition may be between 19.2 N and 77 N at 24° C. to 25° C. and between 31.8 N and 90.5 N at 22° C. to 23° C. In some embodiments, the force required to administer a dose of 80 USP units/1.0 mL of the repository corticotropin pharmaceutical composition may be between 15.2 N and 67.2 N at 24° C. to 25° C. and between 19.6 N and 155.5 N at 22° C. to 23° C. In some embodiments, the method may result in a breakage rate of less than 30% during administration. In some embodiments, the composition may be consistently administered between about 4 to about 8 mm below the surface of the skin of the patient with every performed administration. In some embodiments, the corticotropin pharmaceutical composition may be consistently administered at a skin depth of 4 mm to 8 mm.

### Definitions

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example by reference to a document where the term is originally used). Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.”

The terms “connected” or “coupled” is defined as connected, whether directly or indirectly through intervening components, and is not necessarily limited to physical connections. The connection can be such that the objects are permanently connected or releasably connected.

As used herein, “about” refers to numeric values, including whole numbers, fractions, percentages, etc., whether or not explicitly indicated. The term “about” generally refers to a range of numerical values, for instance, +0.5-1%, +1-5% or +5-10% of the recited value, that one would consider equivalent to the recited value, for example, having the same function or result.

As used herein, “repository corticotropin pharmaceutical composition,” “repository corticotropin injection,” “repository corticotropin,” “corticotropin,” ACTHAR Gel®, “medicine” and “drug product” may be used interchangeably. Acthar Gel® is a naturally sourced complex mixture of adrenocorticotropic hormone analogs and other pituitary peptides. The Acthar Gel® manufacturing process converts the initial porcine pituitary extract with low ACTH content into a mixture having modified porcine ACTH and other related peptide analogs solubilized in gelatin. A major component in the formulated complex mixture is N-25 deamidated porcine ACTH (1-39).

Acthar Gel® is supplied as a sterile preparation in 16% gelatin to provide a prolonged release after intramuscular or subcutaneous injection. Acthar Gel® also contains 0.5% phenol, not more than 0.1% cysteine (added), sodium hydroxide and/or acetic acid to adjust pH and water for injection.

ACTH is a 39 amino acid peptide hormone secreted by the anterior pituitary gland. ACTH is secreted from the anterior pituitary in response to corticotropin-releasing hormone (CRH) that is secreted from the hypothalamus. The release of ACTH stimulates melanocortin receptors in the adrenal cortex with subsequent increased production of glucocorticosteroids and/or cortisol from the adrenal cortex, as well as binding to other melanocortin receptor subtypes.

ACTH is synthesized from a precursor polypeptide pre-pro-opiomelanocortin (pre-POMC). The removal of the signal peptide during translation produces a 267 amino acid polypeptide POMC. POMC undergoes a series of post-translational modifications to yield various polypeptide fragments including and not limited to ACTH, β-lipotropin, γ-lipotropin, α, β, γ-Melanocyte Stimulating Hormone (MSH) and β-endorphin. POMC, ACTH and β-lipotropin are also secreted from the pituitary gland in response to the hormone corticotropin-releasing hormone (CRH). Without being bound to any one particular theory, it is believed that ACTH (e.g, ACTH1-39 or ACTHAR®) or fragments thereof, such as for example, ACTH1-24 (SYNACTHEN®; tetracosactide) or ACTH1-20, can be advantageous in the methods described herein due to the ability to induce a steroidogenic or a partial steroidogenic effect, depending on the peptide chosen and on the dosage regimen. In some embodiments, multiple hypothalamic, pituitary, and peripheral factors regulate stress-mediated or inflammation-induced POMC expression and/or ACTH secretion. Essential cellular functions maintaining metabolic and neuroendocrine control require a homeostatic, non-stressed pattern of ACTH and glucocorticoid secretion. ACTH secretion is characterized by both circadian periodicity and ultradian pulsatility that is generated by CRH release and is also influenced by peripheral corticosteroids. Thus, ACTH secretion peaks at about before 7 am and nadir adrenal steroid secretion occurs between about 11 pm and 3 am, with periodic secretory bursts occurring throughout the day. Serum cortisol levels also exhibit a similar pattern of circadian periodicity. These rhythms are further reinforced by visual cues and the light-dark cycle. In some instances, stress results in increased ACTH pulse amplitude. In some cases stress may be associated with loss of the diurnal rhythms.

The term “ACTH” refers to corticotropin, adrenocorticotropic hormone, Tetracosactide or the like. The term “ACTH” also includes, but is not limited to, any ACTH peptide or any ACTH preparation as described herein. In some embodiments, ACTH is an ACTH peptide. As used herein, in some embodiments, “ACTH peptide” refers to ACTH1-39 peptide of structure:

(SEQ ID NO: 1)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Xₐₐ₁-Gly-Ala-Glu-Asp-Xₐₐ₂-
21 22 23 24 25 26 27 28 29 30
Leu-Ala-Glu-Ala-Phe-Pro-Leu-Glu-Phe-OH
31 32 33 34 35 36 37 38 39

or any homologs, analogs, fragments, complexes or aggregates thereof, wherein Xₐₐ₁ is Asp or Asn; and Xₐₐ₂ is Gln or Glu. The term ACTH includes peptides or peptide fragments, complexes, salts or aggregates with about 40%, about 45%, about 50%, about 55%, about 60%, about 65%, about 70%, about 75%, about 80%, about 85%, about 90%, or about 95% homology with ACTH1-39. The term ACTH includes ACTH from any source including human ACTH, mouse ACTH, rat ACTH, porcine ACTH, sheep ACTH, bovine ACTH, rabbit ACTH or any other source of ACTH.

In some embodiments, the ACTH peptide or fragment, analog, complex, or aggregate thereof, or any combination thereof, is a porcine ACTH peptide or fragment, analog, complex, or aggregate thereof; a human ACTH peptide or fragment, analog, complex, or aggregate thereof; or a recombinant ACTH peptide or fragment, analog, complex, or aggregate thereof. In some embodiments, the ACTH peptide is a porcine ACTH peptide, a human ACTH peptide, or a recombinant ACTH peptide. In some embodiments, the ACTH peptide is a porcine ACTH peptide. In some embodiments, the ACTH peptide is a human ACTH peptide. In some embodiments, the ACTH peptide is a recombinant ACTH peptide. In some embodiments, the ACTH peptide is a recombinant human ACTH peptide.

In some embodiments, ACTH is an ACTH preparation. As used herein, “ACTH preparation” refers to a mixture containing ACTH peptide and/or other peptide fragments and/or other proteins and/or other substances that together form a composition that is suitable for any methods and/or dosing regimen described herein. In some of such embodiments, ACTH is obtained from a homogenized pituitary extract of an appropriate animal (e.g., pituitary extract of a pig). Any suitable method is used to obtain a homogenized pituitary extract. In some of such embodiments, a homogenized pituitary extract includes ACTH peptide and/or other peptide fragments and/or other proteins and/or other substances that are contemplated as being part of the ACTH preparation that is compatible with any method described herein.

“U” or “USP”, when appended to dosage amounts, designates a standardized unit of biologic activity as measured by the USP assay for repository corticotropin injection, which provides uniformity of dosing of a hormone peptide, and is expressed as the standardized units of activity per mL, such as, for example, 80 U/mL.

As used herein, “success rate” means the rate at which a full dose is delivered to the patient.

As used herein, “full dose” or “complete dose” means the full amount of the repository corticotropin injection contained within the pre-filled manual injector for a human patient in need thereof. For example, to receive the full dose, users of the manual injector apparatus have to push the handle straight down until the colored body (green for 40 USP, purple for 80 USP) disappears and the device clicks.

As used herein, “manual apparatus,” “pre-filled manual apparatus,” “pre-filled manual injector apparatus,” “manual injector apparatus,” “injector apparatus,” “manual injector,” “Selfdose device,” “SmartDose,” and “Acthar Delivery Device” are used interchangeably to refer to a device for manual, single-use injection of Acthar Gel.

As used herein, “pediatric patient” refers to a patient ≥2 years of age and <18 years of age.

As used herein, “adult” refers to a person is ≥18 years old.

### Manual Injector Apparatus

Provided herein is a pre-filled manual injector apparatus that is configured to hold a repository corticotropin injection. The pre-filled manual injector apparatus may be operable to administer a one-time use dose only of the repository corticotropin injection to an adult patient in need thereof. In some examples, the adult human patient in need thereof is ≥18 years old.

The pre-filled manual injector apparatus may be configured to inject the repository corticotropin injection intramuscularly or subcutaneously. In an example, the injector apparatus is configured to provide a subcutaneous injection under the skin or into a fat layer of the patient. The manual injector apparatus may allow for the patient to self-administer the dose of the repository corticotropin injection.

In an embodiment, the repository corticotropin injection (RPI) is a naturally sourced complex mixture comprising N-25 deamidated porcine ACTH (1-39). For example, the repository corticotropin injection may be Acthar Gel®.

The pre-filled manual injector apparatus is intended for one-time use such that it contains a single dose of the repository corticotropin injection. The pre-filled manual injector may include a syringe that contains the repository corticotropin injection. The syringe may be pre-filled and may not be removable from the manual injector apparatus. For example, the syringe may be pre-assembled in the manual injector apparatus and may not be re-fillable. The pre-filled manual injector may contain 0.5 mL to 5 mL of the repository corticotropin injection. In at least one example, the pre-filled manual injector may contain 1 mL of 80 USP repository corticotropin injection. In another example, the pre-filled manual injector may contain 0.5 mL of 40 USP repository corticotropin injection. In an embodiment, the pre-filled manual injector comprises a dose of 40 USP or 80 USP of the repository corticotropin injection. In some embodiments, the pre-filled manual injector comprises a dose of 40 USP units/0.5 mL or 80 USP units/1.0 mL of the repository corticotropin injection. After 2 weeks of treatment, dosing may be gradually tapered and discontinued over a 2-week period. In the treatment of acute exacerbations of multiple sclerosis, daily intramuscular or subcutaneous doses of 80-120 units for 2-3 weeks may be administered. In the treatment of other disorders and diseases, dosing may be individualized depending on the disease under treatment and the medical condition of the patient. The dose may be tapered.

The repository corticotropin injection in the pre-filled manual injector apparatus may be stored under refrigeration between 2° C. to 8° C. (36° F. to 46° F.). In some embodiments, the repository corticotropin injection in the pre-filled manual injector apparatus may be warmed to room temperature before using. In an embodiment, the manual injector apparatus may be removed from the refrigerator and set out at room temperature for 30 minutes up to 24 hours before administration of the repository corticotropin injection. In some embodiments, the manual injector apparatus is capable of being stored at room temperature for up to 24 hours prior to administration. In other embodiments, the repository corticotropin injection in the pre-filled manual injector apparatus may be used without warming to room temperature. In additional embodiments, the manual injector apparatus may be removed from the refrigerator and set out at room temperature for at least 45 minutes and up to 24 hours before administration of the repository corticotropin injection.

The repository corticotropin injection may have a higher viscosity, such that it may be more difficult to inject from a syringe than a composition with a viscosity lower than the repository corticotropin injection. In some embodiments, the repository corticotropin injection has a minimum viscosity of 5.00 cPs and maximum viscosity of 30.00 cPs. In other embodiments, the repository corticotropin injection may have a minimum viscosity of 9.85 cPs and a maximum viscosity of 27.05 cPs. For example, the repository corticotropin injection may have a viscosity of at least 5 cPs, at least 10 cPs, at least 15 cPs, at least 20 cPs, at least 25 cPs, or up to 30 cPs. The repository corticotropin injection may have a mean viscosity that is between the minimum viscosity and the maximum viscosity. The mean viscosity may be averaged over a temperature range and over a time period. The temperature of the repository corticotropin injection may change the viscosity of the repository corticotropin injection.

In an embodiment, the pre-filled manual injector apparatus includes a pre-filled syringe. The syringe may include a needle and plunger. In various embodiments, the syringe may include a 23-gauge needle, a 25-gauge needle, or a 27-gauge needle. In some embodiments, the pre-filled manual injector is a transparent syringe that is capable of withstanding the force needed to inject the repository corticotropin injection. In an embodiment, the transparent syringe is plastic or glass.

In an embodiment, the manual injector apparatus may further include a housing. The housing may be configured to contain and completely surround the syringe. The manual injector apparatus may further include a handle, and the handle must be pushed down by hand. The handle may be in contact with or connected to a plunger of the syringe, such that pushing the handle down also presses down the plunger to eject the repository corticotropin injection from the needle of the syringe and into the patient. The manual injector apparatus may further include a needle guard to protect from injury after use. The needle guard may surround the needle and may extend past the end of the needle after the repository injection has been injected into the patient. The manual injector apparatus may further include a window to view at least a portion of the syringe and/or repository corticotropin injection. The manual injector apparatus may further include a cap to cover the needle prior to and after administration of the repository corticotropin injection. In some examples, removing the cap further removes a rigid needle shield over the needle of the syringe. The manual pre-filled apparatus may include an injector that controls the needle insertion depth to deliver medication to dermal tissue at a depth of 4 to 8 mm beneath the outer surface of the skin. The injector may also prevent drug delivery until the needle guard is depressed such that the needle protrudes past the needle guard by at least 4 mm.

In some embodiments, the housing may further include an indicator to indicate a full dose of the repository corticotropin injection has been administered. In an embodiment, the indicator may be a colored portion on the housing operable to be completely covered when the full dose is administered. in another embodiment, the indicator may be an audible click that is triggered when the full dose is administered. In other embodiments, the needle guard may further include a needle guard indicator to show that the needle guard is locked after the repository corticotropin injection has been administered.

Due to the increased viscosity of the repository corticotropin injection, a higher injection force may be needed to eject the repository corticotropin injection from the pre-filled manual injector. The temperature of the repository corticotropin injection may have an effect on the viscosity of the repository corticotropin injection. The manual injector may have a maximum injection force that varies based on the temperature of the repository corticotropin injection. Any drop is viscosity roughly translates to a proportional drop in force. For example, the maximum injection force may be directly proportional to the viscosity of the repository corticotropin injection. In one example for illustration, if a set of apparatuses was known to take 20N to inject and the viscosity of that lot dropped by 10%, that may cause a decrease of about 2N to the injection force when injected at a slower operation force speed of about 150 mm/min and at room temperature. At faster inject speeds and cooler temps, the relationship between viscosity and injection force may be more non-linear. In some embodiments, the maximum injection force of the manual injector is determined by a pancake force gauge.

The maximum injection force to inject the repository corticotropin injection at a temperature of 2° C. and 8° C. may range from about 130 N to about 230 N, about 130 N to about 150 N, about 140 N to about 160 N, about 150 N to about 170 N, about 160 N to about 180 N, about 170 M to about 190 N, about 180 N to about 200 N, about 190 N to about 210 N, about 200 N to about 220 N, and about 210 N to about 230 N. In some examples, the manual injector comprises a maximum injection force of 225.3 N with a 25-gauge needle at a temperature between 2° and 8° C. In another example, the manual injector comprises a maximum injection force of 135.1 N with a 23-gauge needle at a temperature between 2° and 8° C.

The maximum injection force to inject the repository corticotropin injection at a temperature of 24° C. to 25° C. may range from about 15 N to about 80 N, about 15 N to about 40 N, about 30 N to about 50 N, about 40 N to about 60 N, about 50 N to about 70 N, and about 60 N about 80 N. In an example, the force required to administer the pre-filled manual injector comprising a dose of 40 USP units/0.5 mL of repository corticotropin is between 19.2 to 77.0 N at 24° C. to 25° C. In another example, the force required to administer the pre-filled manual injector comprising a dose of 80 USP units/mL of repository corticotropin may be between 15.2 to 67.2 N at 24° C. to 25° C.

The maximum injection force to inject the repository corticotropin injection at a temperature of 22° C. to 23° C. may range from about 18 N to about 160 N, about 18 N to about 40 N, about 30 N to about 50 N, about 40 N to about 60 N, about 50 N to about 70 N, about 60 N about 80 N, about 70 N to about 90 N, about 80 N to about 100 N, about 90 N to about 110 N, about 100 N to about 120 N, about 110 N to about 130 N, about 120 N to about 140 N, about 130 N to about 150 N, and about 140 to about 160 N. In an example, the force required to administer the pre-filled manual injector comprising a dose of 40 USP units/0.5 mL of repository corticotropin may be between 31.8 to 90.5 N at 22° C. to 23° C. In another example, the force required to administer the pre-filled manual injector comprising a dose of 80 USP units/mL of repository corticotropin may be between 19.6 N to 155.5 N at 22° C. to 23° C.

In some embodiments, the manual injector apparatus achieves over a 90% success rate at administering a full dose to a patient in need thereof at a temperature between 22° C. and 25° C. In other embodiments, the manual injector achieves at least a 60% success rate at administering a full dose to a patient in need thereof at any temperature between 2° C. and 25° C. In some embodiments, the manual injector apparatus may achieve a device breakage rate of less than 30%, less than 25%, less than 20%, less than 15%, less than 10%, less than 5%, or less than 1% during administration.

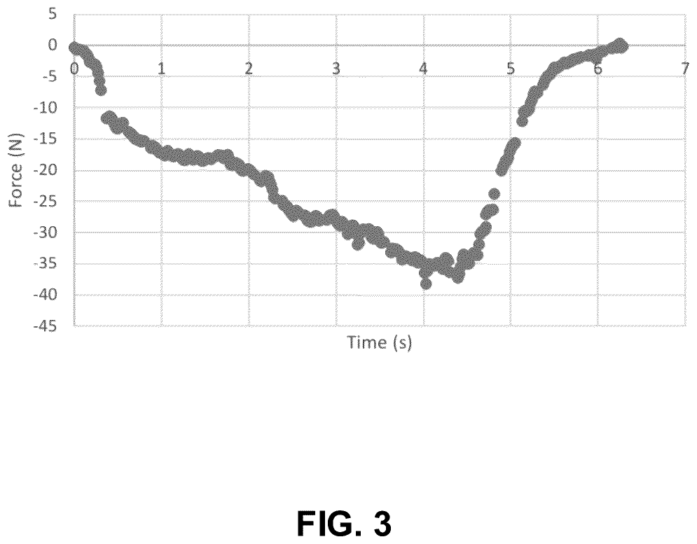

In some embodiments, the manual injector apparatus may be stored in a sealed plastic tray, as seen in FIG. 3. The injector apparatus, in the sealed plastic tray, may then be stored in the refrigerator. Once the injector apparatus is ready to use, the sealed plastic tray may be removed from the refrigerator and the injector apparatus may be removed from the plastic tray. The injector apparatus may remain in the sealed plastic tray while warming to room temperature. The sealed plastic tray may include the dose, the expiration date, medication name, and/or some instructions for use.

The pre-filed manual injector apparatus provides for an injection time of about 5 seconds to 10 seconds, about 5 seconds to 30 seconds, or 2.5 seconds to 50 seconds. In some embodiments, the manual injector apparatus provides for an injection time of at least 2.5 s, 2.5 s to 10 s, 5 s to 15 s, 10 s to 20 s, 15 s to 25 s, 20 s to 30 s, 25 s to 35 s, 30 s to 40 s, 35 s to 45 s, or 40 s to 50 s.

### Methods

Provided herein are methods of injecting a repository corticotropin injection into a patient in need thereof. In some embodiments, the patient in need thereof is an adult patient ≥18 years old. The repository corticotropin injection may be administered as treatment of acute exacerbations of multiple sclerosis in adults, adjunctive therapy for short-term administration (to tide the patient over an acute episode or exacerbation) in: Psoriatic arthritis; Rheumatoid arthritis, including juvenile rheumatoid arthritis (selected cases may require low-dose maintenance therapy); Ankylosing spondylitis, maintenance therapy in selected cases of: systemic lupus erythematosus, systemic dermatomyositis (polymyositis), treatment of severe erythema multiforme, Stevens-Johnson syndrome, serum sickness, severe acute and chronic allergic and inflammatory processes involving the eye and its adnexa such as: keratitis; iritis, iridocyclitis, diffuse posterior uveitis and choroiditis, optic neuritis, chorioretinitis; anterior segment inflammation, symptomatic sarcoidosis, or to induce a diuresis or a remission of proteinuria in the nephrotic syndrome without uremia of the idiopathic type or that due to lupus erythematosus.

In an embodiment, the repository corticotropin injection (RPI) is a naturally sourced complex mixture comprising N-25 deamidated porcine ACTH (1-39). For example, the repository corticotropin injection may be Acthar Gel®.

The pre-filled manual injector apparatus is intended to one-time use such that contains a single dose of the repository corticotropin injection. The pre-filled manual injector may contain 0.5 mL to 5 mL of the repository corticotropin injection. In at least one example, the pre-filled manual injector may contain 1 mL of the repository corticotropin injection. In at least one example, the pre-filled manual injector may contain 0.5 mL of the repository corticotropin injection. In some embodiments, the pre-filled manual injector comprises a dose of 40 USP units/0.5 mL or 80 USP units/1.0 mL of the repository corticotropin injection. After 2 weeks of treatment, dosing may be gradually tapered and discontinued over a 2-week period. In the treatment of acute exacerbations of multiple sclerosis, daily intramuscular or subcutaneous doses of 80-120 units for 2-3 weeks may be administered. In the treatment of other disorders and diseases, dosing may be individualized depending on the disease under treatment and the medical condition of the patient. The dose may be tapered.

In some embodiments, the method may include removing a manual injector apparatus containing a pre-filled manual injector apparatus from a refrigerator. The refrigerator may maintain the repository corticosteroid injection at a temperature between 2° C. and 8° C. After removing the manual injector apparatus from the refrigerator, it may be left at room temperature for between 30 minutes up to 24 hours.

In some embodiments, the manual injector apparatus may be stored in a sealed plastic tray. The injector apparatus, in the sealed plastic tray, may then be stored in the refrigerator. Once the injector apparatus is ready to use, the sealed plastic tray may be removed from the refrigerator and the injector apparatus may be removed from the plastic tray. The injector apparatus may remain in the sealed plastic tray while warming to room temperature. In some examples, the method may include inspecting the expiration date on the sealed plastic tray and not administering the repository corticotropin injection if it is past the expiration date.

In an embodiment, the method may include sitting the manual injector apparatus on a clean, dry, flat surface at room temperature for a minimum of 30 minutes before administering the repository corticotropin injection. In another embodiment, the method may include sitting the injector apparatus on a clean, dry, flat surface at room temperature for a minimum of 60 minutes before administering the repository corticotropin injection.

The method may further include administering the repository corticotropin injection via the pre-filled manual injector apparatus. The repository corticotropin injection may be injected into the patient between 30 minutes up to 24 hours after removal of the from the refrigerator. In other embodiments, the repository corticotropin injection in the pre-filled manual injector apparatus may be used without warming to room temperature.

The repository corticotropin injection may have a higher viscosity, such that it may be more difficult to inject from a syringe than a composition with a viscosity lower than the repository corticotropin injection. In some embodiments, the repository corticotropin injection has a minimum viscosity of 5.00 cPs and maximum viscosity of 30.00 cPs. In other embodiments, the repository corticotropin injection may have a minimum viscosity of 9.85 cPs and a maximum viscosity of 27.05 cPs. For example, the repository corticotropin injection may have a viscosity of at least 5 cPs, at least 10 cPs, at least 15 cPs, at least 20 cPs, at least 25 cPs, or up to 30 cPs. The temperature of the repository corticotropin injection may change the viscosity of the repository corticotropin injection.

Due to the increased viscosity of the repository corticotropin injection, a higher injection force may be needed to eject the repository corticotropin injection from the pre-filled manual injector apparatus. The temperature of the repository corticotropin injection may have an effect on the viscosity of the repository corticotropin injection. The manual injector apparatus may have a maximum injection force that varies based on the temperature of the repository corticotropin injection. For example, the maximum injection force may be directly proportional to the viscosity of the repository corticotropin injection. In some embodiments, the force applied to the manual injector apparatus increases during the administering step.

The maximum injection force to inject the repository corticotropin injection at a temperature of 2° C. and 8° C. may range from about 130 N to about 230 N, about 130 N to about 150 N, about 140 N to about 160 N, about 150 N to about 170 N, about 160 N to about 180 N, about 170 M to about 190 N, about 180 N to about 200 N, about 190 N to about 210 N, about 200 N to about 220 N, and about 210 N to about 230 N. In some examples, the manual injector apparatus requires a maximum injection force of 225.3 N with a 25-gauge needle at a temperature between 2° C. and 8° C. In another example, the manual injector apparatus requires a maximum injection force of 135.1 N with a 23-gauge needle at a temperature between 2° C. and 8° C.

The maximum injection force to inject the repository corticotropin injection at a temperature of 24° C. to 25° C. may range from about 15 N to about 80 N, about 15 N to about 40 N, about 30 N to about 50 N, about 40 N to about 60 N, about 50 N to about 70 N, and about 60 N about 80 N. In an example, the force required to administer a dose of 40 USP units/0.5 mL of repository corticotropin is between 19.2 N and 77.0 N at 24° C. to 25° C. In another example, the force required to administer a dose of 80 USP units/1.0 mL of repository corticotropin may be between 15.2 N and 67.2 N at 24° C. to 25° C.

The maximum injection force to inject the repository corticotropin injection at a temperature of 22° C. to 23° C. may range from about 18 N to about 160 N, about 18 N to about 40 N, about 30 N to about 50 N, about 40 N to about 60 N, about 50 N to about 70 N, about 60 N about 80 N, about 70 N to about 90 N, about 80 N to about 100 N, about 90 N to about 110 N, about 100 N to about 120 N, about 110 N to about 130 N, about 120 N to about 140 N, about 130 N to about 150 N, and about 140 to about 160 N. In an example, the force required to administer a dose of 40 USP units/0.5 mL of repository corticotropin may be between 31.8 N and 90.5 N at 22° C. to 23° C. In another example, the force required to administer a dose of 80 USP units/1.0 mL of repository corticotropin may be between 19.6 N and 155.5 N at 22° C. to 23° C.

In some embodiments, the method may include warming the manual injector to a temperature sufficient to avoid an oscillatory skewed force pattern during the administering step. The administering step may provide an injection force versus time graph curve as depicted in FIG. 3.

In an embodiment, the method may further include inspecting the repository corticotropin injection in an injector apparatus window. The injector apparatus window may allow the patient or other user to see and inspect the repository corticotropin injection. Inspecting the repository corticotropin injection may include inspecting for contamination. In some examples, the repository corticotropin injection may not be administered if cloudiness or small flecks are observed in the repository corticotropin injection through the window.

In an embodiment, the method further includes removing a bottom cap from the manual injector apparatus prior to administering the repository corticotropin injection. In some examples, removing the cap further removes a rigid needle shield over the needle of the syringe.

In another embodiment, the method further includes placing the manual injector apparatus flat on cleaned skin at a 90-degree angle. The skin should not be pinched when placing the manual injector apparatus.

In another embodiment, the method may further include pushing a handle of the manual injector apparatus down to inject the repository corticotropin, without lifting the injector or locking out the injection during administration. Fully pressing down the handle to deliver the full dose of the repository corticotropin injection may trigger an indicator. In an embodiment, the indicator may be a colored portion of the housing of the manual injector apparatus that is fully covered when the full dose is delivered. Disappearance of the color indicator indicates a complete dosing. In some examples, the method may include inspecting the indicator upon completion of administration. In another embodiment, the indicator may be an audible click that is triggered with the full dose is administered. In some examples, the method may further include automatically setting off a click sound generated from the manual injector apparatus upon completion of administration.

In some embodiments, the method may further include injection of the repository corticotropin wherein the repository corticotropin is consistently administered between 4 mm to 8 mm below the surface of the skin of the patient. For example, the repository corticotropin may be consistently administered between about 4 mm to about 8 mm, about 4 mm to about 7 mm, about 4 mm to about 6 mm, about 4 mm to about 5 mm, about 5 mm to about 8 mm, about 5 mm to about 7 mm, about 5 mm to about 6 mm, about 6 mm to about 8 mm, about 6 mm to about 7 mm, or about 7 mm to about 8 mm below the surface of the skin of the patient. In additional examples, the repository corticotropin may be consistently administered between 4 mm to 8 mm, 4 mm to 7 mm, 4 mm to 6 mm, 4 mm to 5 mm, 5 mm to 8 mm, 5 mm to 7 mm, 5 mm to 6 mm, 6 mm to 8 mm, 6 mm to 7 mm, or 7 mm to 8 mm below the surface of the skin of the patient. In other examples, the repository corticotropin may be consistently administered about 4 mm, about 5 mm, about 6 mm, about 7 mm, or about 8 mm below the surface of the skin of the patient.

In an embodiment, the method may further include removing the manual injector apparatus off the patient. In some examples, the removal from the skin of the patient automatically locks a needle guard into place. Locking the needle guard in place may reveal a needle guard indicator to show that the needle guard is locked after the repository corticotropin injection has been administered.

In an embodiment, the method may further include disposing the manual injector apparatus into a sharps container.

In various embodiments, the administering is to an upper thigh, abdomen, or back of arm of the patient. In some examples, the patient may self-administer the repository corticotropin injection to their upper thigh or abdomen using the manual injector apparatus. In other examples, another person may administer the repository corticotropin injection to the patient's back of arm using the manual injector apparatus. The repository corticotropin injection is not administered to a navel, knee, or groin area of the patient.

In an embodiment, the method may further include gathering an alcohol swab, bandage, sharps container, and combinations thereof prior to administering. Then, the method may include cleaning an injection site with the alcohol swab and not touching or fanning the injection site after cleaning. The administering does not occur through clothing and is to an injection site without irritated skin, tattoos, warts, scars, or birthmarks.

### Pediatric Patients

The method may include removing a pre-filled manual apparatus from a refrigerator, the manual apparatus comprising a repository corticotropin pharmaceutical composition for a patient ≥2 years old in need thereof; and administering the repository corticotropin pharmaceutical composition to the patient between 30 minutes and up to 24 hours after removal from the refrigerator by an adult. In some embodiments, the repository corticotropin is administered to a pediatric patient in need by an adult.

In some embodiments, the patient in need thereof is a pediatric patient ≥2 years old. The pediatric patient may be about 2-5 years old, about 3-6 years old, about 4-7 years old, about 5-8 years old, about 6-9 years old, about 7-10 years old, about 8-11 years old, about 9-12 years old, about 10-13 years old, about 11-14 years old, about 12-15 years old, about 13-16 years old, about 14-17 years old, about 15-18 years old, about 2-16 years old, or about 2-17 years old. The pediatric patient may be about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, or about 17 years old.

The repository corticotropin injection may be administered as treatment of juvenile rheumatoid arthritis (selected cases may require low-dose maintenance therapy); maintenance therapy in selected cases of: systemic lupus erythematosus, systemic dermatomyositis (polymyositis), treatment of severe erythema multiforme, Stevens-Johnson syndrome, serum sickness, severe acute and chronic allergic and inflammatory processes involving the eye and its adnexa such as: keratitis; iritis, iridocyclitis, diffuse posterior uveitis and choroiditis, optic neuritis, chorioretinitis; anterior segment inflammation, symptomatic sarcoidosis, or to induce a diuresis or a remission of proteinuria in the nephrotic syndrome without uremia of the idiopathic type or that due to lupus erythematosus.

In an embodiment, the repository corticotropin injection (RPI) is a naturally sourced complex mixture comprising N-25 deamidated porcine ACTH (1-39). For example, the repository corticotropin injection may be Acthar Gel®.

The pre-filled manual injector apparatus is intended to one-time use such that contains a single dose of the repository corticotropin injection. The pre-filled manual injector may contain 0.5 mL to 5 mL of the repository corticotropin injection. In at least one example, the pre-filled manual injector may contain 1 mL of the repository corticotropin injection. In at least one example, the pre-filled manual injector may contain 0.5 mL of the repository corticotropin injection. In some embodiments, the pre-filled manual injector comprises a dose of 40 USP units/0.5 mL or 80 USP units/1.0 ml of the repository corticotropin injection. After 2 weeks of treatment, dosing may be gradually tapered and discontinued over a 2-week period.

In some embodiments, the method may include removing a manual injector apparatus containing a pre-filled manual injector apparatus from a refrigerator. The refrigerator may maintain the repository corticosteroid injection at a temperature between 2° C. and 8° C. After removing the manual injector apparatus from the refrigerator, it may be left at room temperature for between 30 minutes up to 24 hours. The temperature of the repository corticosteroid injection may be between 22° C. to 25° C. The temperature may be from about 22° C. to about 25° C. or about 23° C. to about 24° C.

In some embodiments, the manual injector apparatus may be stored in a sealed plastic tray. The injector apparatus, in the sealed plastic tray, may then be stored in the refrigerator. Once the injector apparatus is ready to use, the sealed plastic tray may be removed from the refrigerator and the injector apparatus may be removed from the plastic tray. The injector apparatus may remain in the sealed plastic tray while warming to room temperature. In some examples, the method may include inspecting the expiration date on the sealed plastic tray and not administering the repository corticotropin injection if it is past the expiration date.

In an embodiment, the method may include sitting the manual injector apparatus on a clean, dry, flat surface at room temperature for a minimum of 30 minutes before administering the repository corticotropin injection. In another embodiment, the method may include sitting the injector apparatus on a clean, dry, flat surface at room temperature for a minimum of 60 minutes before administering the repository corticotropin injection.

The method may further include administering the repository corticotropin injection via the pre-filled manual injector apparatus. The repository corticotropin injection may be injected into the patient between 30 minutes up to 24 hours after removal of the from the refrigerator. In other embodiments, the repository corticotropin injection in the pre-filled manual injector apparatus may be used without warming to room temperature.

In some embodiments, the method may further include injection of the repository corticotropin to a pediatric patient wherein the repository corticotropin is consistently administered between 4 mm to 8 mm below the surface of the skin of the patient. For example, the repository corticotropin may be consistently administered between about 4 mm to about 8 mm, about 4 mm to about 7 mm, about 4 mm to about 6 mm, about 4 mm to about 5 mm, about 5 mm to about 8 mm, about 5 mm to about 7 mm, about 5 mm to about 6 mm, about 6 mm to about 8 mm, about 6 mm to about 7 mm, or about 7 mm to about 8 mm below the surface of the skin of the patient. In additional examples, the repository corticotropin may be consistently administered between 4 mm to 8 mm, 4 mm to 7 mm, 4 mm to 6 mm, 4 mm to 5 mm, 5 mm to 8 mm, 5 mm to 7 mm, 5 mm to 6 mm, 6 mm to 8 mm, 6 mm to 7 mm, or 7 mm to 8 mm below the surface of the skin of the patient. In other examples, the repository corticotropin may be consistently administered about 4 mm, about 5 mm, about 6 mm, about 7 mm, or about 8 mm below the surface of the skin of the patient. In some examples, ultrasound echography may be used to measure skin thickness in the deltoid and suprascapular regions of infants and adolescents.

The repository corticotropin injection may have a higher viscosity, such that it may be more difficult to inject from a syringe than a composition with a viscosity lower than the repository corticotropin injection. In some embodiments, the repository corticotropin injection has a minimum viscosity of 5.00 cPs and maximum viscosity of 30.00 cPs at a temperature of 22° C. to 25° C. at the time of administration. In other embodiments, the repository corticotropin injection may have a minimum viscosity of about 6 cPs and a maximum viscosity of about 29 cPs, about 7 cPs and a maximum viscosity of about 28 cPs, about 8 cPs and a maximum viscosity of about 27 cPs, about 9 cPs and a maximum viscosity of about 26 cPs, about 10 cPs and a maximum viscosity of about 25 cPs, about 11 cPs and a maximum viscosity of about 24 cPs, about 12 cPs and a maximum viscosity of about 23 cPs, about 13 cPs and a maximum viscosity of about 22 cPs, about 14 cPs and a maximum viscosity of about 21 cPs, about 15 cPs and a maximum viscosity of about 20 cPs, about 16 cPs and a maximum viscosity of about 19 cPs, or about 17 cPs and a maximum viscosity of about 18 cPs at a temperature of 22° C. to 25° C. at the time of administration. For example, the repository corticotropin injection may have a viscosity of at least 5 cPs, at least 10 cPs, at least 15 cPs, at least 20 cPs, at least 25 cPs, or up to 30 cPs at a temperature of 22° C. to 25° C. at the time of administration. The temperature of the repository corticotropin injection may change the viscosity of the repository corticotropin injection.

The maximum injection force to inject the repository corticotropin injection at a temperature of 22° C. and 25° C. may range from about 10 N to about 200 N. For example, the maximum injection force to inject the repository corticotropin injection at a temperature of 22° C. and 25° C. may be about 10 N to about 190 N, about 10 N to about 180 N, about 10 N to about 170 N, about 10 N to about 160 N, about 10 N to about 150 N, about 10 N to about 140 N, about 10 N to about 130 N, about 10 N to about 120 N, about 10 N to about 110 N, about 10 N to about 100 N, about 10 N to about 90 N, about 10 N to about 80 N, about 10 N to about 70 N, about 10 N to about 60 N, about 10 N to about 50 N, about 10 N to about 40 N, about 10 N to about 30 N, about 10 N to about 20 N, about 15 N to about 200 N, about 15 N to about 190 N, about 15 N to about 180 N, about 15 N to about 170 N, about 15 N to about 160 N, about 15 N to about 150 N, about 15 N to about 140 N, about 15 N to about 130 N, about 15 N to about 120 N, about 15 N to about 110 N, about 15 N to about 100 N, about 15 N to about 90 N, about 15 N to about 80 N, about 15 N to about 70 N, about 15 N to about 60 N, about 15 N to about 50 N, about 15 N to about 40 N, about 15 N to about 30 N, about 15 N to about 20 N, about 20 N to about 200 N, about 20 N to about 190 N, about 20 N to about 180 N, about 20 N to about 170 N, about 20 N to about 160 N, about 20 N to about 150 N, about 20 N to about 140 N, about 20 N to about 130 N, about 20 N to about 120 N, about 20 N to about 110 N, about 20 N to about 100 N, about 20 N to about 90 N, about 20 N to about 80 N, about 20 N to about 70 N, about 20 N to about 60 N, about 20 N to about 50 N, about 20 N to about 40 N, about 20 N to about 30 N, about 30 N to about 200 N, about 30 N to about 190 N, about 30 N to about 180 N, about 30 N to about 170 N, about 30 N to about 160 N, about 30 N to about 150 N, about 30 N to about 140 N, about 30 N to about 130 N, about 30 N to about 120 N, about 30 N to about 110 N, about 30 N to about 100 N, about 30 N to about 90 N, about 30 N to about 80 N, about 30 N to about 70 N, about 30 N to about 60 N, about 30 N to about 50 N, about 30 N to about 40 N, about 40 N to about 200 N, about 40 N to about 190 N, about 40 N to about 180 N, about 40 N to about 170 N, about 40 N to about 160 N, about 40 N to about 150 N, about 40 N to about 140 N, about 40 N to about 130 N, about 40 N to about 120 N, about 40 N to about 110 N, about 40 N to about 100 N, about 40 N to about 90 N, about 40 N to about 80 N, about 40 N to about 70 N, about 40 N to about 60 N, about 40 N to about 50 N, about 50 N to about 200 N, about 50 N to about 190 N, about 50 N to about 180 N, about 50 N to about 170 N, about 50 N to about 160 N, about 50 N to about 150 N, about 50 N to about 140 N, about 50 N to about 130 N, about 50 N to about 120 N, about 50 N to about 110 N, about 50 N to about 100 N, about 50 N to about 90 N, about 50 N to about 80 N, about 50 N to about 70 N, about 50 N to about 60 N, about 60 N to about 200 N, about 60 N to about 190 N, about 60 N to about 180 N, about 60 N to about 170 N, about 60 N to about 160 N, about 60 N to about 150 N, about 60 N to about 140 N, about 60 N to about 130 N, about 60 N to about 120 N, about 60 N to about 110 N, about 60 N to about 100 N, about 60 N to about 90 N, about 60 N to about 80 N, about 60 N to about 70 N, about 70 N to about 200 N, about 70 N to about 190 N, about 70 N to about 180 N, about 70 N to about 170 N, about 70 N to about 160 N, about 70 N to about 150 N, about 70 N to about 140 N, about 70 N to about 130 N, about 70 N to about 120 N, about 70 N to about 110 N, about 70 N to about 100 N, about 70 N to about 90 N, about 70 N to about 80 N, about 80 N to about 200 N, about 80 N to about 190 N, about 80 N to about 180 N, about 80 N to about 170 N, about 80 N to about 160 N, about 80 N to about 150 N, about 80 N to about 140 N, about 80 N to about 130 N, about 80 N to about 120 N, about 80 N to about 110 N, about 80 N to about 100 N, about 80 N to about 90 N, about 90 N to about 200 N, about 90 N to about 190 N, about 90 N to about 180 N, about 90 N to about 170 N, about 90 N to about 160 N, about 90 N to about 150 N, about 90 N to about 140 N, about 90 N to about 130 N, about 90 N to about 120 N, about 90 N to about 110 N, about 90 N to about 100 N, about 100 N to about 200 N, about 100 N to about 190 N, about 100 N to about 180 N, about 100 N to about 170 N, about 100 N to about 160 N, about 100 N to about 150 N, about 100 N to about 140 N, about 100 N to about 130 N, about 100 N to about 120 N, about 100 N to about 110 N, about 110 N to about 200 N, about 110 N to about 190 N, about 110 N to about 180 N, about 110 N to about 170 N, about 110 N to about 160 N, about 110 N to about 150 N, about 110 N to about 140 N, about 110 N to about 130 N, about 110 N to about 120 N, about 120 N to about 200 N, about 120 N to about 190 N, about 120 N to about 180 N, about 120 N to about 170 N, about 120 N to about 160 N, about 120 N to about 150 N, about 120 N to about 140 N, about 120 N to about 130 N, about 140 N to about 200 N, about 140 N to about 190 N, about 140 N to about 180 N, about 140 N to about 170 N, about 140 N to about 160 N, about 140 N to about 150 N, about 150 N to about 200 N, about 150 N to about 190 N, about 150 N to about 180 N, about 150 N to about 170 N, about 150 N to about 160 N, about 160 N to about 200 N, about 160 N to about 190 N, about 160 N to about 180 N, about 160 N to about 170 N, about 170 N to about 200 N, about 170 N to about 190 N, about 170 N to about 180 N, about 180 N to about 200 N, about 180 N to about 190 N, about 190 N to about 200 N, about 15 N to about 175 N, about 20 N to about 150 N, about 50 N to about 125 N, or about 75 N to about 100 N.

The maximum injection force to inject the repository corticotropin injection at a temperature of 22° C. to 25° C. may range from about 15 N to about 100 N, about 20 N to about 90 N, about 30 N to about 80 N, about 40 N to about 70 N, about 50 N to about 60 N, or about 60 N about 80 N.

In an example, the force required to administer a dose of 40 USP units/0.5 mL of repository corticotropin is between 19.2 N and 77.0 N at 24° C. to 25° C. In an example, the force required to administer a dose of 40 USP units/0.5 mL of repository corticotropin is between 31.8 N and 90.5 N at 22° C. to 23° C.

In another example, the force required to administer a dose of 80 USP units/1.0 mL of repository corticotropin may be between 15.2 N and 67.2 N at 24° C. to 25° C. In another example, the force required to administer a dose of 80 USP units/1.0 mL of repository corticotropin may be between 19.6 N and 155.5 N at 22° C. to 23° C.

The composition may be administered at a delivering rate of 150 mm/min+/−5 mm/min. The delivering rate may be 150 mm/min+/−4 mm/min, 150 mm/min+/−3 mm/min, 150 mm/min+/−2 mm/min, or 150 mm/min+/−1 mm/min. In some examples, the delivering rate may be from about 145 mm/min to about 155 mm/min, about 146 mm/min to about 154 mm/min, about 147 mm/min to about 153 mm/min, about 148 mm/min to about 152 mm/min, about 149 mm/min to about 151 mm/min.

### EXAMPLES

### Example 1

### Participants

Eighteen participants were recruited who experience some level of motor impairment that has an impact on their hands, arms, and/or shoulders. These participants were all recruited from a subset of the patient population of intended users. The following characterizes this group:

Has symptoms from one of the following:
Multiple sclerosis
Rheumatoid Arthritis
Psoriatic Arthritis
Systematic Lupus Erythematous
Ankylosing Spondylitis

Regularly experiences symptoms that impact hands, arms, and/or shoulders, these could be:
Motor impairment
Weakness
Pain
Limited range of motion
Tremors

### Experience Giving Self-Injections

Participants were recruited as two groups, one of which was previously identified participants from prior studies, while the other consisted of new participants. Most participants reported that they experienced weakness, pain, or limited range of motion in their hands, arms, and/or shoulders. Two re-recruited participants did not report these symptoms in the survey, but both did report occasional symptoms.

### Research Stimuli and Equipment

The stimuli used in this study included Acthar Delivery Devices in thermoformed plastic trays sealed by a Tyvek cover as well as equipment to collect force data.

Acthar Delivery Device in individual tray packaging that included:

Four configurations of SmartDose: 40 and 80 USP versions with 23 and 25-gauge needles. All Selfdose device components were platform production mold tool components apart from the Syringe Retainer, Lower Housing and Upper Housing. These three components came off a prototype mold of the new high strength component design. To expedite production of the upper housing component, the tip of the upper housing plunger rod was 3D printed and glued onto the plunger rod. The prototype component accuracy and surface finish was not to production specification.

The 25G syringes used in this build were Ompi glass syringes (SPC-0142 Rev 01). The 23G syringes used in this build were West CZ plastic syringes. The break loose and glide force performance from both syringes was noted to be similar meaning injection force performance should not be affected by the difference in syringe material.

All devices were filled with a worst-case higher-than-average viscosity Acthar.

Thermoformed trays with sealed and labeled Tyvek cover with device and quick reference guide (QRG) inside.

5-UP outer cartons for 40 and 80 USP devices for shipping-participants were not presented with cartons.

Quick Reference Guide (QRG) Document, included in each plastic tray with device.

A force gauge rig included: an injection pad, a force gauge, a PC equipped with LV-1000 software to record force data, a 3D printed injection pad holder, wooden and metal support frames with adjustable height: 1 for abdominal injections and 1 for thigh injections.

Further equipment included a chair, a sharps container, a refrigerator, temperature loggers, a digital camera, a video camera, a multi-channel timer, a laser thermometer (certified as accurate to within 2° C. at the temperatures observed), a moderator's guide, an insight content and release form, and a participant check list. All storage refrigerators were continuously monitored to ensure temperatures from 2° C. to 8° C. were maintained.

### Study Procedure

Participants utilized devices at different temperatures realistic to warming conditions. As part of the set-up, devices were kept in a refrigerator kept from 2° C. to 8° C. to simulate actual temperatures. The interior temperature was continuously monitored for temperature during long-term storage.

Some procedures (including temperature logging) were modifications to the original protocol to support a contactless procedure to protect participants and researchers from potential COVID-19 contamination.

The moderator asked questions about the participant's demographic background. Some questions were directed at the participant's experience using injection devices. The interview aimed to confirm each person's eligibility for participation and provide context for the study data.

The moderator introduced the device by presenting the Acthar delivery device and provided a brief overview of the function without instruction on usage steps. Participants were given a device at room temperature with a QRG and told to prepare to give the injection. Participants were allowed as much time as they needed to feel comfortable giving the injection.

Each participant was asked where they would inject and was assigned a force rig based on this answer, simulating injection into either the abdomen or thigh. They then completed a single trial injection and were asked how successful they were. Participants who had issues during the trial were given instruction to ensure they performed the force test injections correctly. Participants were also encouraged to push a capped device against the injection site to get a feel for how this device would feel pressed against their body for the injection.

Participants were assigned to either inject 40- or 80-USP devices, which they were presented with one at a time. Each participant completed at least six trial injections: 2 warmed for 60 minutes, 2 for 30 minutes, and 2 right out of the fridge. For each warming condition, participants injected one device with a 23-gauge needle and one with a 25-gauge needle. Presentation order of the needle gauges was counterbalanced across participants. Participants were given devices in the order of 60-minute, 30-minute, and 0-minute warming times, with this order modified for some participants to allow them to inject devices closer to the target time. All injectors were left in their respective plastic trays during the warming period.

Whenever possible, devices were administered within 5 minutes of their target time (e.g. 60 minutes has a window from 55 minutes to 65 minutes). Due to the nature of this study, it was not always possible for injections to be completed within this target window.

All injections were performed into an injection pad affixed to a force gauge.

### Detailed Task Descriptions

While preparing for an injection and injecting using the Acthar delivery device, a set of tasks are completed by the user. The tasks that were investigated for the current study were: open packaging-tray, remove contents and device, choose an injection site, remove bottom cap, place device on injection site, push handle straight down, and lift device off the injection site.

Needle gauge presentation order was counterbalanced across participants to control for order effects. For each pair of devices, based on warming condition (60 min, 30 min, 0 min), participants were presented with one 23-gauge device and one 25-gauge, in varying order.

Temperature conditions were controlled as reasonably possible within the confines of this study. Presentation order of temperatures were not counterbalanced, saving the more difficult to inject, colder devices for later in the session. After a room temperature learning/practice injection, trials proceeded with 60 minutes, then 30 minutes and finally 0 minutes out of a refrigerator. Counterbalancing did not occur because recruitment focused on participants with motor impairments, and researchers did not want to exhaust participants with the cold devices and prevent them from being able to complete warmer devices for a more accurate needle gauge comparison.

For the practice injection, participants used an injector that had been removed from the refrigerator and warmed in a room set to 70° F. (21° C.) overnight.

Configuration: All excess components were removed from the table, and the participant was provided with a device in a sealed tray. The participant was asked where they would inject (prompted with “abdomen or thigh” if other location declared), with a force rig including a pancake gauge moved into position based on their declared site. The force rig required some participants to get up to be properly placed. This configuration was utilized for all injections.

Expected actions: open tray lid, remove device from tray, check device label for drug, dose, and expiration date, “wait 30 minutes” (simulated), wash hands, select injection site, clean injection site, inspect liquid window, remove cap, place on pad at 90-degrees, depress needle shield, push handle to inject medication, keep in place for entire duration of injection, notice click-probe, notice colored body-probe, remove from skin after click (lifting straight up), notice yellow line-probe, and dispose in sharps container.

For injections 1 and 2, participants used an injector that had been removed from the refrigerator and warmed in a room set to 70° F. (21° C.) for approximately 60 minutes inside its tray packaging with the exact time sitting out and device temperature recorded. The moderator assessed the temperature of the device using an infrared laser thermometer aimed at the drug window by scanning around the window and recording the lowers registered surface temperature.

Configuration: All excess components were removed from the table, and the participant was provided with a device in a sealed tray. The participant was asked where they would inject (prompted with “abdomen or thigh” if other location declared), with a force rig including a pancake gauge moved into position based on their declared site. The force rig required some participants to get up to be properly placed. This configuration was utilized for all injections.

Expected actions: remove cap, place on pad at 90-degrees, depress needle shield, push handle to inject medication, keep in place for entire duration of injection, notice click—probe, notice colored body—probe, remove from skin after click (lifting straight up), notice yellow line—probe, and dispose in sharps container.

For injections 3 and 4, the process for injections 1 and 2 was repeated, but with a 30-minute warming period as opposed to a 60-minute warming period.

For injections 5 and 6, Participants used an injector that had just been removed from the refrigerator to attempt to give an injection, with the temperature of the refrigerator continuously monitored using a temperature logger. The moderator assessed the temperature of the device using an infrared laser thermometer aimed at the drug window by scanning around the window and recording the lowers registered surface temperature.

Configuration: All excess components were removed from the table, and the participant was provided with a device in a sealed tray. The participant was asked where they would inject (prompted with “abdomen or thigh” if other location declared), with a force rig including a pancake gauge moved into position based on their declared site. The force rig required some participants to get up to be properly placed. This configuration was utilized for all injections.

Expected actions: remove cap, place on pad at 90-degrees, depress needle shield, push handle to inject medication, keep in place for entire duration of injection, notice click-probe, notice colored body-probe, remove from skin after click (lifting straight up), notice yellow line-probe, and dispose in sharps container.

Due to the world-wide outbreak of COVID-19, researchers modified all aspects of interaction with participants. These modifications were far-reaching and had implications for the study including more minor changes such as requiring researchers and participants to wear a mask and gloves, and not providing participants with a sharps container and other supplies. There were some more impactful changes as well, notably separating the researcher to a separate room (impacted timing and photography) and reducing building personnel while requiring more extensive check-ins including a health screening (impacted timing by delaying some start times).

Additionally, the study dates were delayed by approximately 4 months, which resulted in the Acthar Delivery Devices requiring long-term storage.

### Use Scenario: Practice Injection

Participants' initial introduction to the device was without any explanation on how it functioned or how they should use it. This allowed researchers to gauge their understanding and correct use utilizing only the QRG. Note that some participants were specifically recruited who had participated in a prior study utilizing prototypes of this device.

Success on tasks was recorded during the practice injection, with participants typically successful overall. In part, this high success can be attributed to the fact that not only did all 14 participants have injection experience of some kind, but 5 of them (P01, P05, P09, P14, P15) were re-recruits who had used this same device in a previous study. Two tasks, “choose an injection site” and “push handle straight down” are discussed further.

The Acthar Delivery Device supports subcutaneous injection into the thigh and the abdomen. Each participant for the present study was asked where they would inject, and their chosen sites were used. All 14 participants selected a site, with an even split between the thigh (n=7) and the abdomen (n=7).

To receive the full dose, users of the Acthar Delivery Device have to push the handle straight down until the colored body (green for 40 USP, purple for 80 USP) disappears and the device clicks. Participants were reminded to push as if they were pushing on themselves to inject the medication (and not just push on the device as hard as they could). This is considered a critical task and has been explored in prior formatives.

Out of 14 participants, 1 participant (P16) failed to complete the injection the first time and was assigned a use error. When asked if she had completed the injection, P16 said “I did not”. Her explanation was that she “didn't know how long to hold it down”. Like several other participants, P16 was on Humira, which uses an injector that requires participants to hold down the pen after clicking a button on top. The pen automatically administers the medication, which is different than SmartDose—a device that functions more like a syringe where users are actively pushing out medication. This error, therefore, is attributed to negative transfer from using a system like Humira. When provided with a second device to practice with, P16 was able to use it successfully. The device that was not completed was 80 USP and contained a 25-gauge needle.

### Use Scenario: Devices Warmed for 60 Minutes

Every participant injected at least two devices warmed inside the plastic tray for approximately 60 minutes, with six participants (P01, P03, P04, P05, P17, P18) injecting 4 each. Altogether, 40 devices were injected for this group.

All 40 devices were successfully injected, regardless of needle gauge. Overall characteristics of these injections can be seen in Table 1. Total counts weigh towards 80 USP devices because extra participants each completed additional trials with 80 USP devices after their 40 USP trials.

TABLE 1
60 Minute Characteristics

| | Averages by Dose | |
|---|---|---|
| Variable | 40 USP (n = 12) | 80 USP (n = 28) |
| Peak Force (N) | 38.2 (SD = 17.6) | 43.8 (SD = 14.8) |
| Max/Min Force | 77.0 to 19.2 | 67.2 to 15.2 |
| Time Warmed | 59.2 (SD = 3.9) | 60.8 (SD = 5.8) |
| (min) | | |
| Temperature (° C.) | 24.6 (SD = 1.0) | 24.2 (SD = 1.1) |
| Inj. Time (s) | 8.8 (SD = 6.6) | 9.0 (SD = 4.5) |

### Use Scenario: Devices Warmed for 30 Minutes

Every participant injected at least two devices warmed inside the plastic tray for approximately 30 minutes. One participant (P13) injected 3 devices at this temperature, and seven participants (P01, P03, P04, P05, P15, P17, P18) injected 4. In total, 43 devices were injected for this group.

Overall characteristics of these injections can be seen in Table 2. Total counts weigh towards 80 USP devices because extra participants each completed additional trials with 80 USP devices after their 40 USP trials.

TABLE 2
30 Minute Characteristics

| | Averages by Dose | |
|---|---|---|
| Variable | 40 USP (n = 14) | 80 USP (n = 29) |
| Peak Force (N) | 56.6 (SD = 15.1) | 73.2 (SD = 32.9) |
| Max/Min Force | 90.5 to 31.8 | 155.5 to 19.9 |
| Time Warmed | 28.3 (SD = 3.7) | 30.5 (SD = 6.1) |
| (min) | | |
| Temperature (° C.) | 23.0 (SD = 1.7) | 22.3 (SD = 1.4) |
| Inj. Time (s) | 9.4 (SD = 3.5) | 11.0 (SD = 4.7) |

There were a total of 4 injections for devices warmed at 30 minutes that were not completed. Each of the 4 were from a different participant: P04, P15, P17, P18. Out of these devices, three (P04, P17, P18) were 40 USP while one (P15) contained 80 USP. There was about a 91% success rate delivering using a device warmed for 30 minutes.

It is important to note that all 4 incomplete injections at this warming condition contained a 25-gauge needle, meaning that for the present study all participants were successful injecting with the 23-gauge device when it was warmed per the instructions.

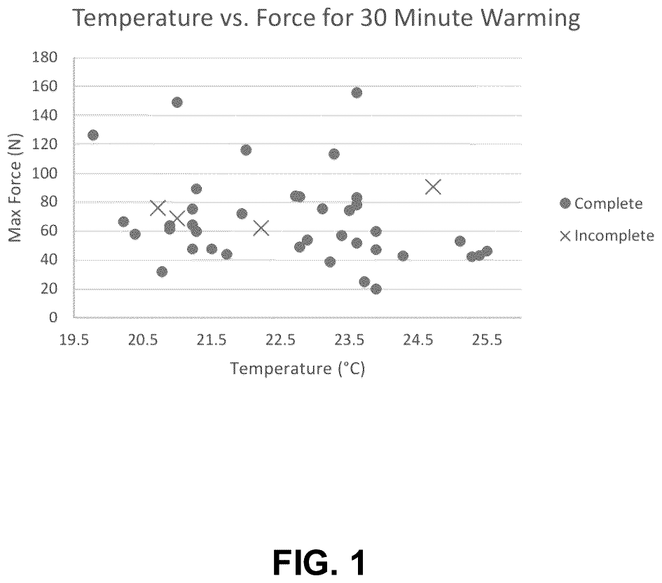

All devices from the 30 minute group are displayed in FIG. 1. Interestingly, all four incompletes had a similar peak force. Of these four participants, three of them (P04, P15, P17) failed complete at least one cold injection. The fourth (P18) was successful with both cold injections.

### Use Scenario: Cold Devices

Because of the potential strain and/or discomfort from attempting to inject cold devices, each participant was only given 2 to inject for each session. This resulted in a total of 28 devices.

Overall characteristics of these injections can be seen in Table 3.

TABLE 3
Cold Device Characteristics

| | Averages by Dose | |
|---|---|---|
| Variable | 40 USP (n = 12) | 80 USP (n = 16) |
| Peak Force (N) | 118.5 (SD = 31.3) | 118.8 (SD = 42.8) |
| Max/Min Force | 183.2 to 79.9 | 225.3 to 69.0 |
| Time Warmed (min) | 0 (SD = 0) | 0 (SD = 0) |
| Temperature (° C.) | 10.7 (SD = 1.4) | 10.4 (SD = 2.2) |
| Inj. Time (s) | 10.7 (SD = 4.2) | 22.8 (SD = 11.9) |

The cold devices were very difficult for participants to inject with. All told, a total of 10 out of the 28 devices were not fully injected. This included one device each from 2 participants (P13, P15), and two devices from 4 participants (P04, P09, P12, P17). The devices were an even split of 23 (n=5) and 25 (n=5) gauge needles. There was about a 65% success rate delivering using a cold device.

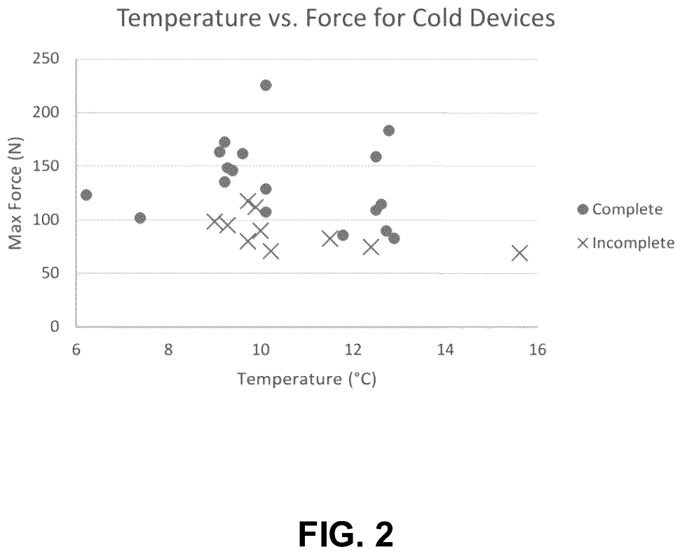

All devices from the 0 minute group are displayed in FIG. 2.

For the cold devices, all incomplete injections were towards the lower end of maximum force exerted. In fact, the five lowest max forces were not completed, with four of these being 25-gauge needles. Because recruitment focused on individuals with motor impairments from the patient population, and all participants had already completed several injections successfully, this finding suggests that this population may actually not always be capable of the amount of force required to inject with a cold device—at least in a timely enough manner that they don't grow fatigued or give up on the injection.

### Force and Time

The forces observed over the course of this study are shown in Table 4, broken down by needle gauge and warming period. Because the gauge is unknown, this table does not include data from the first 8 sessions.

TABLE 4
Peak Forces Observed (in N)

| Time Group | Statistic | 23-gauge | 25-gauge |
|---|---|---|---|
| 60 min | Group Size | n = 20 | n = 20 |
| | Average | 36.3 (SD = 13.8) | 47.9 (SD = 15.8) |
| | (Standard | | |
| | Deviation) | | |
| | Range | 62.9 to 15.17 | 77.0 to 20.91 |
| 30 min | Group Size | n = 22 | n = 20 |
| | Average | 52.4 (SD = 14.2) | 84.4 (SD = 32.4) |
| | (Standard | | |
| | Deviation) | | |
| | Range | 82.9 to 19.88 | 155.5 to 24.78 |
| 0 min | Group Size | n = 14 | n = 14 |
| | Average | 104.2 (SD = 17.8) | 133.1 (SD = 13.8) |
| | (Standard | | |
| | Deviation) | | |
| | Range | 135.1 to 74.65 | 225.3 to 88.95 |

Because the force was recorded regularly over the course of the injection, patterns could be generated to observe how forces were exerted. Interestingly, across participants the pattern observed was skewed towards the end of the injection (shown in FIG. 3), where participants were slowly increasing their force over the injection time until right before the peak force when they would rapidly lift up. These cleaner patterns tended to be shorter injections and could be representative of participants recognizing progress being made on the injection and pushing steadily harder to ensure it was completed.

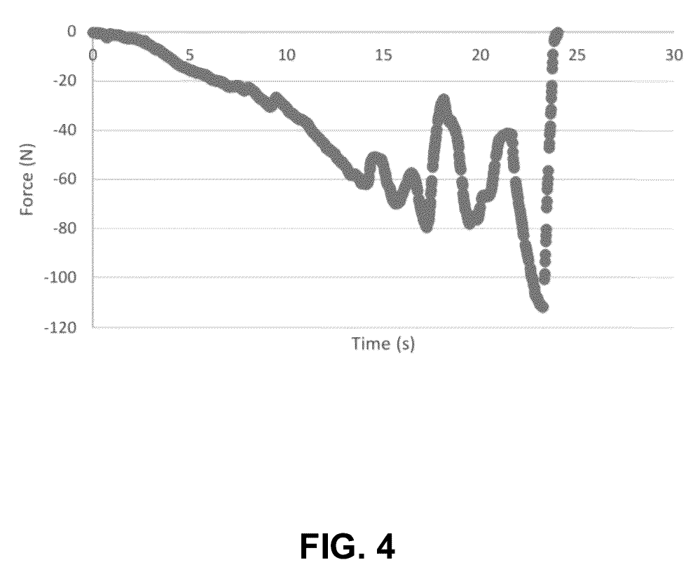

With some participants, this skew became more oscillatory, because participants would ease up and then push with increasing force repeatedly over the course of the injection, creating a series of increasing peak forces that formed a single valley. This pattern became more common with colder, longer injections, as participants grew more fatigued and had to ease up to adjust their grip or gather their strength. In some cases this even included the addition of their other hand. This is shown in FIG. 4, where the first oscillations occurred at about 15 seconds, with oscillations increasing in wave height.

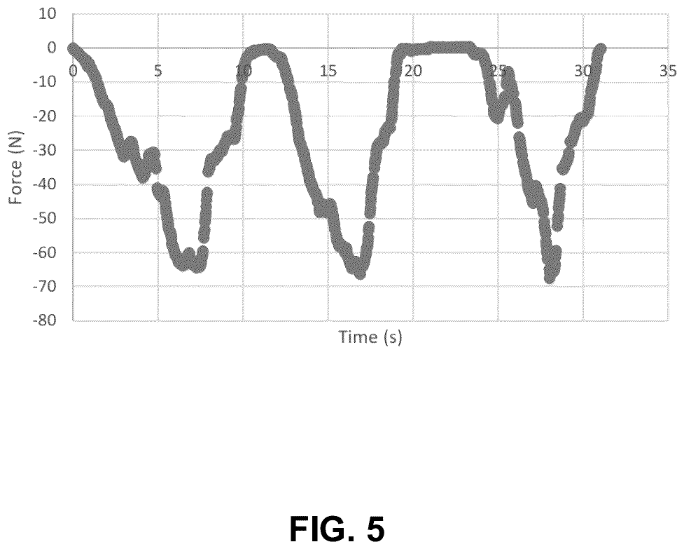

The third main type of force pattern observed, shown in FIG. 5, is characterized by more extreme oscillations that changed the force exerted all the way to zero. In these rarer cases, the participant had a full rests between peaks, which in this case involved P09 lifting the injector completely off the injection pad to inspect it. It should be noted, however, that in this instance P09 did not lock the needle guard prematurely, because while injecting with the cold 80 USP device she was not exerting enough force to move the handle at all.

For the present study, “Injection Time” was defined as the time when a participant started applying force after the cap was removed until they lifted the device off the pad after ending the injection attempt. The reason for this operational definition was to ensure a more accurate understanding of the forces exerted, because often participants continued to push the device even after it clicked, and because of the nature of the force curves it is possible the peak force exerted occurred after the medicine was fully administered.

TABLE 5
Injection Time (in seconds)

| Time Group | Statistic | 23-gauge | 25-gauge |
|---|---|---|---|
| 60 min | Group Size | n = 20 | n = 20 |
| | Average | 8.09 (SD = 4.66) | 9.76 (SD = 5.63) |
| | (Standard | | |
| | Deviation) | | |
| | Range | 2.89 to 19.07 | 3.24 to 26.45 |
| 30 min | Group Size | n = 22 | n = 20 |
| | Average | 9.65 (SD = 3.97) | 11.41 (SD = 4.74) |
| | (Standard | | |
| | Deviation) | | |
| | Range | 4.22 to 17.96 | 3.02 to 22.41 |
| 0 min | Group Size | n = 14 | n = 14 |
| | Average | 15.66 (SD = 11.22) | 19.62 (SD = 10.73) |
| | (Standard | | |
| | Deviation) | | |
| | Range | 2.54 to 48.90 | 5.00 to 49.56 |

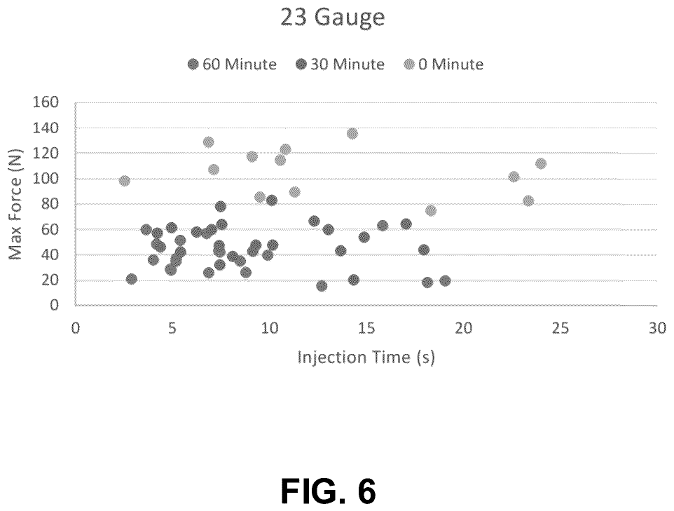

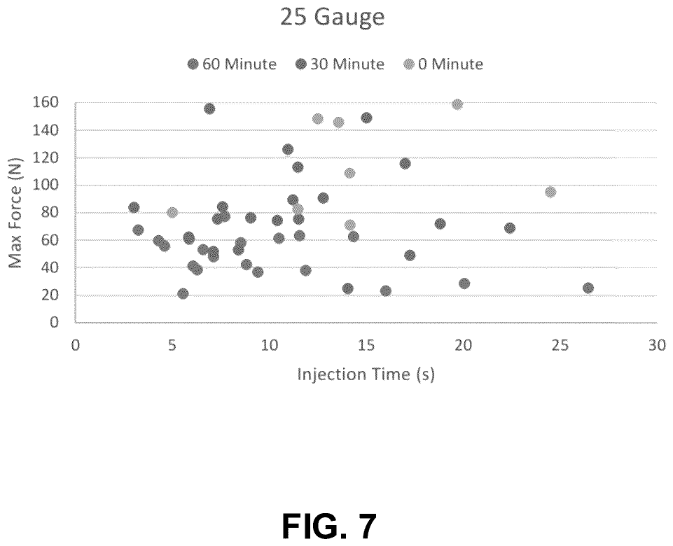

The graphs in FIG. 6 (for 23-gauge) and FIG. 7 (for 25-gauge) show injection time against the maximum force for that injection. In both cases a single extreme outlier is not visible.

Because these graphs include devices from all warming conditions, they demonstrate the trend that injection time and max force have a positive relationship. In addition, they show how this is related to warming condition, with devices from the longer warming condition tending to be closer to zero for both axes.

After every pair of trials, participants were asked if they noticed a difference between the 23-gauge and 25-gauge devices. They were also asked if they had any preference between them. The majority of participants preferred the 23-gauge needle, especially when comparing devices that had been warmed.

Not a single participant expressed preference for a 25-gauge device for warmed devices, with just one participant (P17) who expressed that the 25 g device was easier at the 60 minute warming time. At the 30 minute warming time, all participants (n=14) preferred the 23 gauge device, with the only missing data from P04 who stated preference for the 23 g 80 USP, but did not state a preference for the 40 USP. For the devices right out of the fridge, of the participants who stated a preference (n=10), almost half preferred a 25 g device (P09, P15, P17, P04).

Regardless of the preferred device, the reason for the preference was because the preferred devices was easier to inject, with most participants specifically expressing that their preferred device took less force to inject.

### Example 2

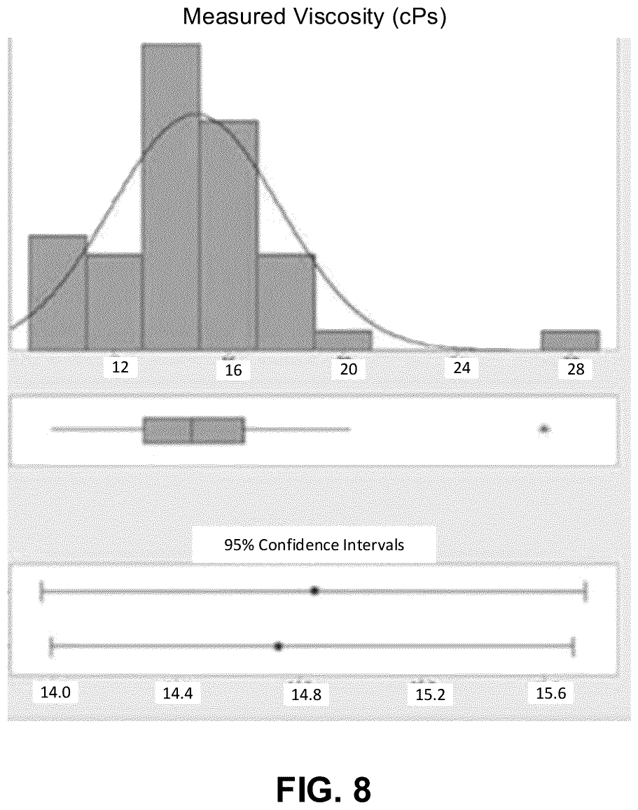

The viscosity of a lot of 46 samples of Acthar Gel was measured at room temperature. FIG. 8 shows the measured viscosity of the lot and the 95% confidence interval. Table 6 below provides specific details about the measured viscosities.

TABLE 6
Anderson-Darling Normality Test

| A-Squared | 0.67 |
|---|---|
| P-Value | 0.077 |
| Mean | 14.848 |
| StDev | 2.971 |
| Variance | 8.828 |
| Skewness | 1.40204 |
| Kurtosis | 5.17451 |
| N | 46 |
| Minimum | 9.855 |
| 1^{st} Quartile | 13.047 |
| Median | 14.732 |
| 3^{rd} Quartile | 16.513 |
| Maximum | 27.050 |
| 95% Confidence Interval for Mean | |
| 13.966 | 15.730 |
| 95% Confidence Interval for Median | |
| 13.994 | 15.689 |
| 95% Confidence Interval for StDev | |
| 2.464 | 3.742 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 3: Clinical Assessment of the Use of a Novel Single-Dose Prefilled Injection Device for the Administration of Acthar Gel in Children

Acthar® Gel (repository corticotropin injection) is a complex mixture of porcine adrenocorticotrophic hormone analogs and other pituitary peptides formulated in a sterile 16% gelatin preparation for extended release after intramuscular (IM) or subcutaneous (SC) injection. Acthar is indicated for the treatment of various inflammatory disorders, including infantile spasms, acute exacerbations of multiple sclerosis, rheumatoid arthritis, systemic lupus erythematosus, symptomatic sarcoidosis, certain nephrotic syndromes and ophthalmic diseases. In pediatric patients, Acthar has been shown to be safe and effective for the treatment of infantile spasms and for diuresis and remission of proteinuria in nephrotic syndrome.

Methods of administration of Acthar Gel using a pre-filled manual apparatus were evaluated for suitability for use in patients under 18 years of age. Factors such as skin depth, needle length and gauge, dosage, force required for injection and potential harms were evaluated.

During evaluation of methods of administering Acthar Gel using a manual prefilled injector, it was determined that younger children may have a higher risk for unintentional (intramuscular) IM injection with their smaller skin-to-muscle ratios. The method of administering using the manual prefilled injector targets a (subcutaneous) SC administration at a depth of 4 mm to 8 mm. Since, there may also be a risk of IM injection in older pediatric groups, particularly if the needle length increased beyond 4 mm, the method of administering using the manual prefilled injector also used an injector with a 4 mm needle. The use of a 4 mm needle in administration resulted in greater safety for all children to avoid inadvertent IM medication delivery. In some methods of administering, in patients aged 2- to 6-years old, the method was used in tandem with the pinch-up technique at all injection sites, even with a 4-mm needle, to increase the skin-to-muscle distance. Furthermore, the method of administering using the prefilled manual injector apparatus was administered perpendicular to the skin.

The pre-filled manual apparatus includes an injector which controlled the needle insertion depth when delivering medication to dermal tissue at a depth of 4 to 8 mm beneath the outer surface of the skin. The injector also prevented drug delivery until the needle guard was depressed during administration such that the needle protrudes past the needle guard by at least 4 mm. A needle length ≥4 mm can carry a risk of IM injection in children, particularly when given perpendicular to the skin. However, Acthar has been shown to be bioequivalent when administered IM or SC. It has been demonstrated that there was no statistically significant difference in total serum cortisol levels between SC or IM administration of identical doses of Acthar. This implies that pre-filled manual apparatus does not pose any additional risks beyond those associated with potential IM injection.

The methods of administering Acthar gel to pediatric patients using a manual prefilled apparatus also included using a 23-gauge needle in the apparatus. Pre-filled manual apparatus used a 0.5-inch (12.7-mm), 23-gauge, thin wall needle (3 bevel) which is appropriate for administration to pediatric patients.

### Force

There is a lack of data on the minimum force required for injection device use in pediatric patients and estimating the lower range of force for this age group is challenging. However, it can be assumed that smaller and lighter patients may require less force for successful SC injection.

When using the pre-filled manual apparatus, the peak axial force to deliver Acthar Gel at a rate of 150±5 mm/min (nominal 15 s dispense) was ≤35 N, a force comparable to inserting a plug into an outlet, when used at 20 to 25° C.

Pull force and dexterity required to remove the Tyvek lid and cap were measured.

Users' ability to remove the cap of the manual prefilled injection device was studied. In the study, 29 patients (including adolescents [n=6], adults [n=11] and elders [n=12]), 6 caregivers and 7 healthcare professionals were tasked with removing the protective cap of a nonfunctional, mock-up injection device with different target cap removal forces between 25 N and 55 N. All participants were able to effectively remove the injection device caps, regardless of sex, age, dexterity impairments, or professional education.

However, the perceived ease of uncapping decreased with higher cap removal force and dexterity impairment, with some participants visibly struggling to remove the caps with the highest cap removal forces.

The tensile force required to remove the pre-filled manual apparatus cap was between 4 N and 35 N. Given that 6 adolescents and other participants with severe dexterity impairments were able to remove the injection device caps in the study, it was anticipated that pediatric patients would be able to remove the pre-filled manual apparatus cap, should they be allowed to use Pre-filled manual apparatus in the future.

The pull force required to remove the pre-filled manual apparatus sealed lid from the tray was between 0.25 and 1.5 pounds of force.

Pre-filled manual apparatus was appropriate for use in pediatric patients if administered by an adult (18 years of age and older) caregiver, family member, or HCP. Needle length and gauge were fitted for this age group, although infants and young children were at the highest risk for unintentional IM injection because of smaller skin-to-muscle distances. Dosage was appropriate for children over 2 years of age, but not for infants and children under 2 years of age. Few studies have shown that adolescents can achieve the minimum force threshold to complete self-injections and remove an injection device cap, but there is a lack of data pertaining to force requirements of injection devices in children. Aside from the potential adverse reactions from Acthar itself, injection site-related AEs were the main concern with pre-filled manual apparatus use.

Numerous examples are provided herein to enhance the understanding of the present disclosure. A specific set of statements are provided as follows.

Statement 1: A pre-filled manual apparatus comprising a repository corticotropin pharmaceutical composition for an adult patient ≥18 years old in need thereof to deliver a one-time use dose only, wherein the apparatus is capable of being stored at room temperature for up to 24 hours prior to administration, and wherein the repository corticotropin pharmaceutical composition is a naturally sourced complex mixture comprising N-25 deamidated porcine ACTH (1-39).

Statement 2: The manual apparatus of statement 1, wherein the manual apparatus is a manual injector apparatus.

Statement 3: The manual apparatus of statement 1, wherein the one-time use dose is 40 USP units/0.5 mL or 80 USP units/1.0 mL of the repository corticotropin pharmaceutical composition.

Statement 4: The manual apparatus of statement 1, wherein the repository corticotropin pharmaceutical composition has a minimum viscosity of 5.00 cPs and maximum viscosity of 30.00 cPs.

Statement 5: The manual apparatus of statement 1, further comprising a transparent syringe that is capable of withstanding a force needed to inject the repository corticotropin pharmaceutical composition.

Statement 6: The manual apparatus of statement 5, wherein the transparent syringe is plastic or glass.

Statement 7: The manual apparatus of statement 5, wherein the repository corticotropin pharmaceutical composition is pre-filled in the transparent syringe.

Statement 8: The manual apparatus of statement 5, wherein the transparent syringe comprises a 23-gauge, 25-gauge, or 27-gauge needle.

Statement 9: The manual apparatus of statement 8, wherein the manual apparatus comprises a maximum injection force of 225.3 N with a 25-gauge needle at a temperature between 2° C. and 8° C.

Statement 10: The manual apparatus of statement 8, wherein the manual apparatus comprises a maximum injection force of 135.1 N with a 23-gauge needle at a temperature between 2° C. and 8° C.

Statement 11: The manual apparatus of statement 1, wherein a force required to administer a dose of 40 USP units/0.5 mL of the repository corticotropin pharmaceutical composition is between 19.2 N and 77.0 N at 24° C. to 25° C.

Statement 12: The manual apparatus of statement 1, wherein a force required to administer a dose of 80 USP units/1.0 mL of the repository corticotropin pharmaceutical composition is between 15.2 N and 67.2 N at 24° C. to 25° C.

Statement 13: The manual apparatus of statement 1, wherein a force required to administer a dose of 40 USP units/0.5 mL of the repository corticotropin pharmaceutical composition is between 31.8 N and 90.5 N at 22° C. to 23° C.

Statement 14: The manual apparatus of statement 1, wherein a force required to administer a dose of 80 USP units/1.0 mL of the repository corticotropin pharmaceutical composition is between 19.6 N and 155.5 N at 22° C. to 23° C.

Statement 15: The manual apparatus of statement 9-14, wherein maximum injection force of the manual apparatus is determined by a pancake force gauge.

Statement 16: The manual apparatus of statement 1, wherein the manual apparatus achieves over a 90% success rate at administering a full dose to the patient in need thereof at any temperature between 22° C. and 25° C.

Statement 17: The manual apparatus of statement 1, wherein the manual apparatus achieves at least a 60% success rate at administering a full dose to the patient in need thereof at any temperature between 2° C. and 25° C.

Statement 18: The manual apparatus of statement 1, wherein the manual apparatus achieves a device breakage rate less than 10% during administration.

Statement 19: The manual apparatus of statement 1, further comprising a handle, wherein the handle must be pushed down by hand.

Statement 20: The manual apparatus of statement 1, wherein the manual apparatus is configured to provide a subcutaneous injection under skin or into a fat layer.

Statement 21: The manual apparatus of statement 1, further comprising a needle guard to protect from injury after use.

Statement 22: The manual apparatus of statement 1, further comprising a housing and an indicator to indicate a full dose of the repository corticotropin pharmaceutical composition has been administered.

Statement 23: The manual apparatus of statement 22, wherein the indicator comprises a colored portion on the housing operable to be completely covered when the full dose is administered and/or an audible click that is triggered when the full dose is administered.

Statement 24: The manual apparatus of statement 1, wherein the manual apparatus is packaged in a sealed plastic tray.

Statement 25: The manual apparatus of statement 1, wherein the manual apparatus is designed for an injection time of about 5 to 10 seconds.

Statement 26: The manual apparatus of statement 1, wherein the manual apparatus is designed for an injection time of about 5 to 30 seconds.

Statement 27: The manual apparatus of statement 1, wherein the manual apparatus is designed for an injection time of about 2.5 to 50 seconds.

Statement 28: A method comprising: removing a pre-filled manual apparatus from a refrigerator, the manual apparatus comprising a repository corticotropin pharmaceutical composition for an adult patient ≥18 years old in need thereof; and administering the repository corticotropin pharmaceutical composition to the patient between 30 minutes and up to 24 hours after removal from the refrigerator, wherein the repository corticotropin pharmaceutical composition is a naturally sourced complex mixture comprising N-25 deamidated porcine ACTH (1-39).

Statement 29: The method of statement 28, wherein the repository corticotropin pharmaceutical composition has a minimum viscosity of 5.00 cPs and maximum viscosity of 30.00 cPs.

Statement 30: The method of statement 28, wherein the administering step comprises pushing a handle of the manual apparatus with a maximum injection force that is directly proportional to the viscosity of the repository corticotropin pharmaceutical composition.

Statement 31: The method of statement 28, wherein the administering step comprises pushing a handle of the manual apparatus with a maximum injection force of 225.3 N with a 25-gauge needle at a temperature between 2° C. and 8° C., wherein the maximum injection force prevents damage to the manual apparatus.

Statement 32: The method of statement 28, wherein the administering step comprises pushing a handle of the manual apparatus with a maximum injection force of 135.1 N with a 23-gauge needle at a temperature between 2° C. and 8° C., wherein the maximum injection force prevents damage to the manual apparatus.

Statement 33: The method of statement 28, wherein the administering step comprises pushing a handle of the manual apparatus with an average injection force between 4 N and 35 N with a 25-gauge needle or a 23-gauge needle.

Statement 34: The method of statement 28, further comprising sitting the manual apparatus on a clean, dry, flat surface at room temperature for a minimum of 30 minutes before administering.

Statement 35: The method of statement 28, further comprising sitting the manual apparatus on a clean, dry, flat surface at room temperature for a minimum of 60 minutes before administering.

Statement 36: The method of statement 28, wherein a force applied to the manual apparatus increases during the administering step.

Statement 37: The method of statement 28, further comprising warming the manual apparatus to a temperature sufficient to avoid an oscillatory skewed force pattern during the administering step.

Statement 38: The method of statement 28, wherein the administering step provides an injection force versus time graph curve as depicted in FIG. 3.

Statement 39: The method of statement 28, further comprising inspecting the repository corticotropin pharmaceutical composition in an injector window.

Statement 40: The method of statement 28, further comprising removing a bottom cap from the manual apparatus prior to administering.

Statement 41: The method of statement 28, further comprising placing the manual apparatus flat on cleaned skin at a 90-degree angle, wherein the skin is not pinched.

Statement 42: The method of statement 28, further comprising pushing a handle of the manual apparatus down to inject the repository corticotropin pharmaceutical composition, without lifting the manual apparatus or locking out the injection during administration.

Statement 43: The method of statement 28, further comprising automatically setting off a click sound generated from the manual apparatus upon completion of administration.

Statement 44: The method of statement 28, further comprising inspecting a color indicator on a housing of the manual apparatus upon completion of administration.

Statement 45: The method of statement 44, wherein disappearance of the color indicator indicates a complete dosing.

Statement 46: The method of statement 28, further comprising removing the manual apparatus off the patient, wherein the removal from the skin of the patient automatically locks a needle guard into place.

Statement 47: The method of statement 28, further comprising disposing the manual apparatus into a sharps container.

Statement 48: The method of statement 28, where in the administering is to an upper thigh, abdomen, or back of arm of the patient.

Statement 49: The method of statement 28, further comprising gathering an alcohol swab, bandage, sharps container, and combinations thereof prior to administering.

Statement 50: The method of statement 28, further comprising cleaning an injection site with an alcohol swab and not touching or fanning the injection site after cleaning.

Statement 51: The method of statement 28, where in the administering does not occur through clothing.

Statement 52: The method of statement 28, where in the administering is to an injection site without irritated skin, tattoos, warts, scars, or birthmarks.

Statement 53: The method of statement 28, where in the administering is not a navel, knee, or groin area of the patient.

Statement 54: The method of statement 28, wherein the manual apparatus is a manual injector apparatus.

Statement 55: A method comprising removing a pre-filled manual apparatus from a refrigerator, the manual apparatus comprising a repository corticotropin pharmaceutical composition for a patient ≥2 years old in need thereof; and administering the repository corticotropin pharmaceutical composition to the patient between 30 minutes and up to 24 hours after removal from the refrigerator; wherein the repository corticotropin pharmaceutical composition is administered by applying a force to the manual apparatus; wherein the repository corticotropin pharmaceutical composition is a naturally sourced complex mixture comprising N-25 deamidated porcine ACTH (1-39); wherein the repository corticotropin pharmaceutical composition has a viscosity of 5 cPs to 30 cPs and a temperature of 22° C. to 25° C. at the time of the administering; wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.; wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.; wherein the method results in a breakage rate of less than 30% during administration; and wherein the composition is consistently administered between 4 to 8 mm below the surface of the skin of the patient.

Statement 56: The method of statement 55, wherein the composition is administered to a pediatric patient ≥2 years of age by an adult of at least 18 years of age.

Statement 57: The method of statement 55, wherein the composition is administered to a pediatric patient ≥2 years of age by an adult of at least 18 years of age.

Statement 58: The method of statement 55, wherein the composition is administered to an adult patient >18 years of age.

Statement 59: The method of statement 55, wherein the force required to administer the dose of 40 USP units/0.5 mL of the repository corticotropin pharmaceutical composition is less than or equal to 35 N.

Statement 60: The method of statement 59, wherein the composition is administered at a rate of 150+/−5 mm/min.

Statement 61: The method of statement 59, wherein the composition is administered at a rate of 150+/−4 mm/min.

Statement 62: The method of statement 59, wherein the composition is administered at a rate of 150+/−3 mm/min.

Statement 63: The method of statement 59, wherein the composition is administered at a rate of 150+/−2 mm/min.

Statement 64: The method of statement 59, wherein the composition is administered at a rate of 150+/−1 mm/min.

Statement 65: The method of statement 55, wherein the force required to administer the dose of 80 USP units/1.0 mL of the repository corticotropin pharmaceutical composition is less than or equal to 35 N.

Statement 66: The method of statement 65, wherein the composition is administered at a rate of 150+/−5 mm/min.

Statement 67: The method of statement 65, wherein the composition is administered at a rate of 150+/−4 mm/min.

Statement 68: The method of statement 65, wherein the composition is administered at a rate of 150+/−3 mm/min.

Statement 69: The method of statement 65, wherein the composition is administered at a rate of 150+/−2 mm/min.

Statement 70: A method of consistent administration of a repository corticotropin pharmaceutical composition comprising removing a pre-filled manual apparatus from a refrigerator, the manual apparatus comprising a repository corticotropin pharmaceutical composition for a patient ≥2 years old in need thereof; and waiting between 45 minutes and up to 24 hours after apparatus removal from the refrigerator; wherein the repository corticotropin pharmaceutical composition is administered by applying a force to the manual apparatus; wherein the repository corticotropin pharmaceutical composition is administered consistently at a skin depth of 4 mm to 8 mm; wherein the repository corticotropin pharmaceutical composition has a viscosity of 5 cPs to 30 cPs and a temperature of 22° C. to 25° C. at the time of the administering; wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.; wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.; and wherein the composition is consistently administered at a skin depth of 4 to 8 mm with every performed administration.

Statement 71: The method of statement 70, wherein the composition is administered to a pediatric patient ≥2 years of age by an adult of at least 18 years of age.

Statement 72: The method of statement 70, wherein the composition is administered to an adult patient >18 years of age.

Statement 73: The method of statement 70, wherein the repository corticotropin pharmaceutical composition is a naturally sourced complex mixture comprising N-25 deamidated porcine ACTH (1-39).

Statement 74: The method of statement 73, wherein the administering step comprises pushing a handle of the manual apparatus with a maximum injection force that is directly proportional to the viscosity of the repository corticotropin pharmaceutical composition.

Statement 75: The method of statement 73, wherein the method results in a breakage rate of less than 30% during administration.

Statement 76: The method of statement 73, wherein the force required to administer the dose of 40 USP units/0.5 mL of the repository corticotropin pharmaceutical composition is less than or equal to 35 N.

Statement 77: The method of statement 76, wherein the composition is administered at a rate is 150+/−5 mm/min.

Statement 78: The method of statement 76, wherein the composition is administered at a rate is 150+/−4 mm/min.

Statement 79: The method of statement 76, wherein the composition is administered at a rate is 150+/−3 mm/min.

Statement 80: The method of statement 76, wherein the composition is administered at a rate is 150+/−2 mm/min.

Statement 81: The method of statement 76, wherein the composition is administered at a rate is 150+/−1 mm/min.

Statement 82: The method of statement 73, wherein the force required to administer the dose of 80 USP units/1.0 mL of the repository corticotropin pharmaceutical composition is less than or equal to 35 N.

Statement 83: The method of statement 82, wherein the composition is administered at a rate of 150+/−5 mm/min.

Statement 83: The method of statement 82, wherein the composition is administered at a rate of 150+/−4 mm/min.

Statement 84: The method of statement 82, wherein the composition is administered at a rate of 150+/−3 mm/min.

Statement 85: The method of statement 82, wherein the composition is administered at a rate of 150+/−2 mm/min.

Statement 86: The method of statement 82, wherein the composition is administered at a rate of 150+/−1 mm/min.

What is claimed is:

1. A method comprising:
removing a pre-filled manual apparatus from a refrigerator, the manual apparatus comprising a repository corticotropin pharmaceutical composition for a patient ≥2 years old in need thereof; and
administering the repository corticotropin pharmaceutical composition to the patient between 30 minutes and up to 24 hours after removal from the refrigerator;
wherein the repository corticotropin pharmaceutical composition is administered by applying a force to the manual apparatus;
wherein the repository corticotropin pharmaceutical composition is a naturally sourced complex mixture comprising N-25 deamidated porcine ACTH (1-39);
wherein the repository corticotropin pharmaceutical composition has a viscosity of 5 cPs to 30 cPs and a temperature of 22° C. to 25° C. at the time of the administering;
wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.;
wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.;
wherein the method results in a breakage rate of less than 30% during administration; and
wherein the composition is consistently administered between 4 to 8 mm below the surface of the skin of the patient.

2. The method of claim 1, wherein the composition is administered to a pediatric patient ≥2 years of age by an adult of at least 18 years of age.

3. The method of claim 1, wherein the composition is administered to an adult patient >18 years of age.

4. The method of claim 1, wherein the force required to administer the dose of 40 USP units/0.5 mL of the repository corticotropin pharmaceutical composition is less than or equal to 35 N.

5. The method of claim 4, wherein the composition is administered at a rate of 150+/−5 mm/min.

6. The method of claim 4, wherein the composition is administered at a rate of 150+/−4 mm/min.

7. The method of claim 4, wherein the composition is administered at a rate of 150+/−3 mm/min.

8. The method of claim 4, wherein the composition is administered at a rate of 150+/−2 mm/min.

9. The method of claim 1, wherein the force required to administer the dose of 80 USP units/1.0 mL of the repository corticotropin pharmaceutical composition is less than or equal to 35 N.

10. The method of claim 9, wherein the composition is administered at a rate of 150+/−5 mm/min.

11. The method of claim 9, wherein the composition is administered at a rate of 150+/−4 mm/min.

12. The method of claim 9, wherein the composition is administered at a rate of 150+/−3 mm/min.

13. The method of claim 9, wherein the composition is administered at a rate of 150+/−2 mm/min.

14. A method of consistent administration of a repository corticotropin pharmaceutical composition comprising:
removing a pre-filled manual apparatus from a refrigerator, the manual apparatus comprising a repository corticotropin pharmaceutical composition for a patient ≥2 years old in need thereof; and
waiting between 45 minutes and up to 24 hours after apparatus removal from the refrigerator;
wherein the repository corticotropin pharmaceutical composition is administered by applying a force to the manual apparatus;
wherein the repository corticotropin pharmaceutical composition is administered consistently at a skin depth of 4 mm to 8 mm;
wherein the repository corticotropin pharmaceutical composition has a viscosity of 5 cPs to 30 cPs and a temperature of 22° C. to 25° C. at the time of the administering;
wherein the force required to administer a dose of 40 USP units/0.5 mL of the repository corticotropin pharmaceutical composition is between 19.2 N and 77 N at 24° C. to 25° C. and between 31.8 N and 90.5 N at 22° C. to 23° C.;
wherein the force required to administer a dose of 80 USP units/1.0 mL of the repository corticotropin pharmaceutical composition is between 15.2 N and 67.2 N at 24° C. to 25° C. and between 19.6 N and 155.5 N at 22° C. to 23° C.; and
wherein the composition is consistently administered at a skin depth of 4 to 8 mm with every performed administration.

15. The method of claim 14, wherein the composition is administered to a pediatric patient ≥2 years of age by an adult of at least 18 years of age.

16. The method of claim 14, wherein the composition is administered to an adult patient >18 years of age.

17. The method of claim 14, wherein the repository corticotropin pharmaceutical composition is a naturally sourced complex mixture comprising N-25 deamidated porcine ACTH (1-39).

18. The method of claim 17, wherein the administering step comprises pushing a handle of the manual apparatus with a maximum injection force that is directly proportional to the viscosity of the repository corticotropin pharmaceutical composition.

19. The method of claim 17, wherein the method results in a breakage rate of less than 30% during administration.

20. The method of claim 17, wherein the force required to administer the dose of 40 USP units/0.5 mL of the repository corticotropin pharmaceutical composition is less than or equal to 35 N.

21. The method of claim 20, wherein the composition is administered at a rate is 150+/−5 mm/min.

22. The method of claim 20, wherein the composition is administered at a rate is 150+/−4 mm/min.

23. The method of claim 20, wherein the composition is administered at a rate is 150+/−3 mm/min.

24. The method of claim 20, wherein the composition is administered at a rate is 150+/−2 mm/min.

25. The method of claim 17, wherein the force required to administer the dose of 80 USP units/1.0 mL of the repository corticotropin pharmaceutical composition is less than or equal to 35 N.

26. The method of claim 25, wherein the composition is administered at a rate of 150+/−5 mm/min.

27. The method of claim 25, wherein the composition is administered at a rate of 150+/−4 mm/min.

28. The method of claim 25, wherein the composition is administered at a rate of 150+/−3 mm/min.

29. The method of claim 25, wherein the composition is administered at a rate of 150+/−2 mm/min.


SEQUENCE LISTING (1 sequence)

SEQ ID NO: 1 - protein, 39 aa, unidentified
SYSMEHFRWGKPVGKKRRPVKVYPDGAEDQLAEAFPLEF